(19) United States
(12) Patent Application Publication
(10) Pub. No.: US 2012/0046232 A1
(43) Pub. Date: Feb. 23, 2012

(54) COMPOSITIONS AND METHODS FOR REDUCING RELAPSE OF ADDICTIVE BEHAVIOR

(75) Inventors: Peter Kalivas, Mt. Pleasant, SC (US); Khaled Moussawi, Charleston, SC (US); Robert Malcolm, Mt. Pleasant, SC (US)

(73) Assignee: Medical University of South Carolina

(21) Appl. No.: 13/166,483

(22) Filed: Jun. 22, 2011

Related U.S. Application Data
(60) Provisional application No. 61/357,363, filed on Jun. 22, 2010.

(51) Int. Cl. A61K 38/06; A61K 31/4439; A61K 31/655; A61K 31/4174; A61K 31/4436; A61P 25/30; A61K 31/60; A61K 31/426; A61K 31/495; A61K 31/4985; A61K 38/05; A61P 25/36; A61K 31/44; A61K 31/4418

(52) U.S. Cl. 514/17.5; 514/277; 514/342; 514/150; 514/398; 514/336; 514/161; 514/369; 514/255.02; 514/249; 514/252.1; 514/252.11

(57) ABSTRACT

Disclosed are methods and compositions comprising mGluR modulators and procysteine drugs. The methods and compositions can be used to treat subjects with prior addiction in order to reduce drug use, drug seeking or relapse.

### CROSS REFERENCE TO RELATED APPLICATIONS

This application claims benefit of and priority to U.S. Provisional Application Ser. No. 61/357,363 filed in the United States Patent and Trademark Office on Jun. 22, 2010; which is incorporated herein by reference in its entirety.

### STATEMENT REGARDING FEDERALLY SPONSORED RESEARCH

This invention was made with government support under DA015369 awarded by the National Institutes of Health. The government has certain rights in the invention.

### BACKGROUND

Recent advances in the elucidation of the neurophysiological roles of mGluRs have established these receptors as promising drug targets in the therapy of acute and chronic neurological and psychiatric disorders and chronic and acute pain disorders. Because of the physiological and pathophysiological significance of the mGluRs, there is a need for new drugs and compounds that can modulate mGluR function.

Addiction and additive behavior continues to plague individuals. While numerous advances in psychosocial and pharmaco therapies have been made, prevention or reduction of relapse of addictive behavior continues to remain elusive. Disclosed are compositions and methods for reducing the relapse rate of addictive behaviors.

### SUMMARY

Disclosed herein are methods of treating subjects comprising, administering a metabotropic glutamate receptor (mGluR) modulator and a procysteine drug, wherein the subject has had a prior addiction

Disclosed herein are methods of inhibiting drug seeking comprising identifying a subject at risk for drug use and administering a mGluR modulator and a procysteine drug to the subject.

Disclosed herein are methods of preventing drug use in a subject comprising identifying the subject as being at risk for drug use and administering a mGluR modulator and a procysteine drug to the subject.

Disclosed herein are methods of decreasing glutamate release in the neuronal synapse and glutamate binding to mGluR5 comprising administering a mGluR modulator and a procysteine drug to a subject at risk for drug use.

Disclosed herein are compositions comprising a mGluR modulator and procysteine drug.

In some forms of the disclosed compositions and methods, the prior addiction can be a drug addiction. The drug addiction can be cocaine addiction.

In some forms of the disclosed compositions and methods, the combination of these compounds (the mGluR modulator and procysteine drug) can reduce drug use.

In some forms of the disclosed compositions and methods, the mGluR modulator can be a negative modulator. In some forms, the negative modulator can be a negative allosteric modulator.

In some forms of the disclosed compositions and methods, the mGluR can be mGluR5. In some forms of the disclosed compositions and methods, the mGluR5 modulator can be a negative modulator. In some forms, the negative mGluR5 modulator can be a negative allosteric modulator. In some forms, the mGluR5 modulator can be 2-methyl-6-(phenylethynyl)pyridine (MPEP). In some forms, the mGluR5 modulator can be 3-((2-Methyl-4-thiazolyl)ethynyl)pyridine (MTEP). In some forms, the mGluR5 modulator can be 1-(3-chlorophenyl)-3-(3-methyl-5-oxo-4H-imidazol-2-yl)urea.

In some forms of the disclosed compositions and methods, the procysteine drug can be N-acetylcysteine (NAC). The NAC can activate mGluR2/3.

In some forms of the disclosed compositions and methods, the mGluR modulator is administered at subthreshold levels. In some forms, the procysteine drug is administered at subthreshold levels. And in some forms, the mGluR modulator and procysteine drug can be both administered at subthreshold levels.

In some forms of the disclosed compositions and methods, the mGluR modulator and procysteine drug can be each administered at therapeutic levels.

In some forms of the disclosed compositions and methods, the mGluR modulator and procysteine drug can have a synergistic effect.

In some forms of the disclosed compositions and methods, the mGluR modulator and procysteine drug can be administered simultaneously. In some forms, the mGluR modulator and procysteine drug can be administered consecutively.

In some forms of the disclosed compositions and methods, the mGluR modulator and procysteine drug can be administered before the subject encounters a drug cue.

In some forms of the disclosed compositions and methods, the mGluR modulator and procysteine drug can be administered intraperitoneally.

In some forms of the disclosed compositions and methods, the mGluR modulator and procysteine drug are administered in one composition, such as a formulation.

In some forms of the disclosed compositions and methods, side effects can be decreased in subjects administered both the mGluR modulator and procysteine drug compared to subjects administered either of them alone.

In some forms of the disclosed compositions and methods, the drug use can be cocaine use.

In some forms of the disclosed compositions and methods, the mGluR modulator can have the structure of:

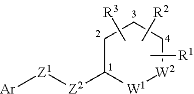

wherein the six membered ring defined by W¹, W² and carbon atoms 1, 2, 3 and 4, can be aromatic or non-aromatic, and further wherein any two neighboring atoms of this six membered ring can be singly or doubly bonded to one another; Z¹ and Z² are either carbon or nitrogen, further wherein Z¹ and Z² can be singly, doubly, or triply bonded to one another and wherein Z² and carbon atom 1 can be singly or doubly bonded to one another, provided that the bond between Z¹ and Z² is not triple when Z₁ and Z₂ are nitrogen, further provided that the bond between Z¹ and Z² is single when the bond between Z² and carbon atom 1 is double; Ar is substituted or unsubstituted aryl or substituted or unsubstituted heteroaryl; one of either W¹ and W² is nitrogen and the other is carbon; R¹, R², and R³ are independently hydrogen, hydroxy, amino, cyano, halo, nitro, mercapto, or a heteroatom-substituted or heteroatom-unsubstituted alkyl, alkenyl, alkynyl, aryl, aralkyl, acyl, alkoxy, alkylamino, or =0; or pharmaceutically acceptable salts, hydrates, tautomers, acetals, ketals, hemiacetals, hemiketals, or optical isomers thereof.

In some forms of the disclosed compositions and methods, Z₁ and Z₂ can be both carbon triply bonded to each other. In some forms, Z₁ and Z₂ can be both nitrogen doubly bonded to each other.

In some forms of the disclosed compositions and methods, Ar can be phenyl, substituted phenyl, thiazole or substituted thiazole.

In some forms of the disclosed compositions and methods, W¹ is nitrogen and W² can be carbon. In some forms, W² can be nitrogen and W¹ can be carbon.

In some forms of the disclosed compositions and methods, R¹, R², and R³ can be independently hydrogen, amino, alkyl, alkenyl or halo.

In some forms of the disclosed compositions and methods, the mGluR modulator can be: phenazopyridine; SIB 1893; SIB 1757; 2-methyl-6-(phenylethynyl)-pyridine (MPEP); NSC41777; 6-methyl-3-phenyldiazenylpyridin-2-amine; 2,6-Diamino-3-(4-iodophenylazo)pyridine; phenyldiazenylpyridin-2-amine; 3-(4-chlorophenyl)diazenylpyridine-2,6-diamine; 3-(2-chlorophenyl)diazenylpyridine-2,6-diamine; 3-[(2-methyl-1,3-thiazol-4-yl)ethynyl]pyridine (MTEP); 1-(3-chlorophenyl)-3-(3-methyl-5-oxo-4H-imidazol-2-yl)urea;

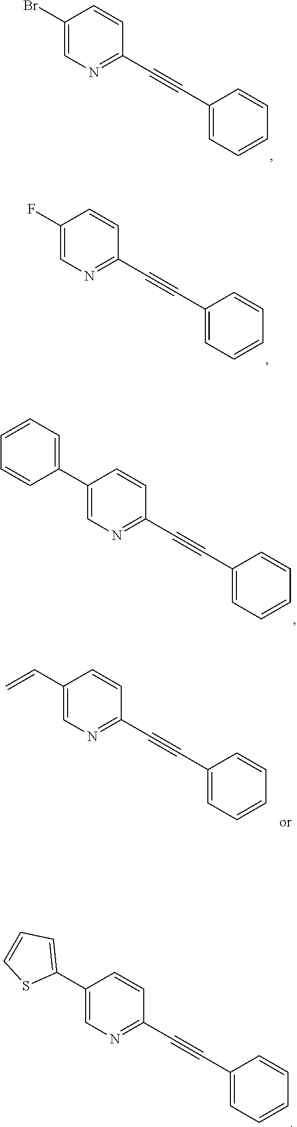

a physiologically acceptable salt thereof; or any mixture thereof.

In some forms of the disclosed compositions and methods, the procysteine drug can be NAC, DiNAC; N-acetylcysteine L-lysine; Carbocisteine; glutathione; S-nitroso-N-acetylcysteine; S-nitrosothiol-N-acetylcysteine; S-allyl-cysteine; S-alkyl-cysteine; N-acetyl-S-farnesyl-cysteine; N-acetyl-L-arginine-NAC; N-acetyl-L-lysine-NAC; N-acetyl-L-histidine-NAC; N-acetyl-L-ornithine-NAC; thioester of NAC with salicylic acid; 2′4′-difluoro-4-hydroxy-(1,1′-diphenyl)-3-carboxylic derivatives of NAC; S-allymercapto-NAC (ASSNaC); N,N-diacetyl-L-cystine; N—S-diacyl-cysteine; N-acetylcysteine conjugate of phenethyl isothiocyanate (PEITC-NAC); S-carboxylmethyl-L-cysteine; derivatives of reacting a reactive derivative of p-isobutylphenylpropionic acid and NAC (e.g., an amide); paraisobutyl NAC; L-2-oxothiazolidine-4-carboxylic acid and a combination thereof.

In some forms of the disclosed compositions and methods, the composition can reduce drug use. In some forms, the mGluR modulator and procysteine drug can be each at subthreshold levels. In some forms, the mGluR modulator and procysteine drug can be each at therapeutic levels. In some forms, the mGluR modulator can be at subthreshold levels and the procysteine drug can be at therapeutic levels. In some forms, the mGluR modulator can be at therapeutic levels and the procysteine drug can be at subthreshold levels. In some forms of the disclosed compositions and methods, side effects can be decreased compared to therapeutic levels of either compound alone.

### BRIEF DESCRIPTION OF THE DRAWINGS

FIG. 1 shows the active lever presses results for when the mGluR5 agonist CHPG was microinjected into the nucleus accumbens of rats trained to self-administer cocaine then withdrawn for 3-5 weeks. Relapse was induced by presenting the animal with a tone and light compound cue that was previously associated with cocaine delivery during the self-administration training. CHPG produces a dose-dependant increase in cue-induced relapse, although alone it did not induce relapse. Data were analyzed using one-way ANOVA (2,14)=5.20, p=0.021; *p<0.05 compared to extinction, +p<0.05 comparing cue+aCSF (artificial cerebrospinal fluid control injection).

FIG. 2 shows the mGluR5 modulator synergizes with NAC to inhibit cocaine relapse. Animals were trained to self-administer cocaine (Self-ad), then extinguished (Ext). Relapse was induced by presenting the tone plus light conditioned cue. Data were evaluated using a one-way ANOVA F(5.34)=10.53, p<0.01; *p<0.05 compared to extinction, +p<0.05 comparing NAC+MTEP to Saline+Saline (Sa+Sa).

FIG. 3 shows that the mGluR5 modulator reduces relapse in animals treated chronically with NAC, but not in animals treated chronically with saline. The left panel shows the basal rate of lever pressing during self-administration and extinction training. The right panel shows that MPEP reduced lever pressing (measure of relapse) in the NAC, but not saline treated animals. Data were analyzed by a two-way ANOVA interaction F(1,20)=7.17, p=0.015; *p<0.05 comparing saline to MPEP.

FIG. 4 is a schematic diagram of extracellular glutamate pools and regulation. The schematic shows the high affinity system X_{AG-} and low affinity system XC⁻ regulation of extracellular glutamate pools.

FIG. 5 shows N-acetylcysteine metabolism and transport. N-acetylcysteine (NAC) is transported into the brain via active transport, most probably through a cysteine transporter. NAC spontaneously oxidizes to cystine and activates system XC⁻ to regulate extracellular glutamate levels. Alternatively, NAC can be transported into glia to be cleaved into cysteine/cystine, which are then transported to the extracellular space to activate system XC⁻. Question marks (?) are used to indicate the lack of supporting evidence. Modified from Holdiness (1991) (Holdiness, 1991).

FIG. 6 shows low doses of mGluR5 antagonists MPEP and Fenobam selectively inhibit relapse in rats that received N-acetylcysteine 10 hours after extinction. Top, treatment protocol for cocaine, N-acetylcysteine, and mGluR5 antagonists. Bottom, low doses of MPEP (1.0 mg/kg/ip) or Fenobam (1.75 mg/kg/ip) inhibit relapse to cocaine seeking only in cocaine rats previously receiving N-acetylcysteine treatment after extinction training (One way ANOVA F(2, 28)=7.922, p=0.0021). *p<0.05, comparing cocaine+cue induced reinstatement in MPEP and Fenobam groups to vehicle group using a Dunnett's post hoc test.

### DETAILED DESCRIPTION

Cys-glut activation activates both mGluR2/3 and mGluR5 (Moussawi et al. Nature Neuroscience 2009). In animal models, both activation of cystine-glutamate exchange and mGluR5 modulators inhibit addictive behaviors. Moreover, pilot clinical trials with cys-glut activation have proven effective at inhibiting addictive behaviors in cocaine, marijuana and nicotine addicts. A cellular mechanistic linkage has been discovered between the two mechanisms of action and these studies demonstrate that the combination of the two drugs suppressing cocaine relapse in animal models is far better than either compound alone. Briefly, this occurs because the cys-glut activation produces two effects, one that inhibits relapse (stimulation of mGluR2/3) and one that promotes relapse (mGluR5 stimulation). Thus, depending on how the animal is treated cys-glut activation is not effective at preventing relapse unless an mGluR5 blocker is co-administered. The data provided herein, shows the utility of co-administration of a cys-glut activation and mGluR5 modulator.

### A. Addiction

Addiction is a chronic brain disease manifested by humans in a variety of behaviors and in a range of social circumstances. Although it is a complex phenomenon, its medical definition is a central nervous system (CNS) disorder manifested as a behavioral disturbance due to a neurobiological imbalance in the brain (Leshner, 1997, Science 278, 45). Individuals may become addicted to a wide variety of factors, including drugs, gambling, alcohol and sex. The obsessive and compulsive aspect of drug dependence may overlap with other obsessive compulsive behaviors such as gambling or compulsive sexual activity.

In respect of substance abuse, addict behavior is induced and maintained in a multifactorial fashion with a central role played by the unconditioned reinforcing properties of the abused drug. There are many different substances on which individuals may become dependent, including opiates, benzodiazepines, amphetamine, nicotine, cocaine and ethanol.

The impact of substance dependence is huge. For example, nicotine dependence is the most widely diffused type of drug addiction. One third of the worldwide population over 15 years of age are smokers. Smoking continues to increase among adolescents and by the year 2025 the WHO estimates that there will be 10 million tobacco related deaths per year. Stopping smoking may evoke a range of symptoms in dependent individuals, including craving, depression, anxiety, difficulty in concentrating and weight gain. Despite a variety of available treatments many smokers fail to give up smoking.

There is therefore a major unmet need in the area of substance abuse for pharmacological agents that are more effective that those currently available at reducing withdrawal symptoms and more importantly reducing relapse rates or drug use. Indeed smoking cessation is a therapeutic area with generally poor results: an average 30% success rate compared with 50 to 80% for alcoholism, opioid and cocaine dependence (at 6 months).

Nevertheless the rationale for pharmacological intervention is, however, still strong because only pharmacotherapy potentially acts on a population larger than that treated with psychosocial interventions and therefore may enhance these traditional methods by improving compliance and quality of the treatment.

Addictive metabotropic glutamate disorders include, for example, nicotine addiction, alcohol addiction, opiate addiction, amphetamine addiction, cocaine addiction, methamphetamine addiction, and the like.

1. Cocaine Addiction

Cocaine Addiction continues to be a devastating problem for individuals, societies, and governments. In addition to addicts' dysfunctions in different aspects of everyday life, cocaine addiction is associated with a constellation of medical complications including increased risks of sexually transmitted diseases like HIV and hepatitis (Booth et al., 1993), increased criminal activities (Conaboy, 1995), neurobehavioral deficits in infants of addicted mothers (Singer et al., 2002), strokes and myocardial infarctions (Brust and Richter, 1977; Cregler and Mark, 1986), and multiple systemic cardiovascular complications (Siegel et al., 1999; Heesch et al., 2000). Cocaine blocks monoamine reuptake transporters resulting in increased levels of norepinephrine, serotonin, and dopamine in the brain. In particular, increased dopamine levels in the nucleus accumbens shell are thought to primarily underlie the rewarding sensation caused by cocaine use (Di Chiara et al., 2004; Everitt and Robbins, 2005). Repeated exposures to cocaine results in cocaine addiction which is characterized by the development of tolerance, withdrawal, loss of interest in usually rewarding activities, and most importantly increased vulnerability to relapse and compulsive drug-seeking upon exposure to the drug itself, stressors, or drug related cues, even after prolonged periods of abstinence (Jaffe et al., 1989; McLellan et al., 1992; Mendelson and Mello, 1996; Dackis and O'Brien, 2001).

Importantly, there is still no approved therapy for cocaine addiction to date. This is partially attributed to the historical perception that addiction is a self-destructive behavior of choice, as is still currently advocated by influential people like Thomas Szasz and John Booth Davies. However, massive evidence from the scientific community in the last 20 years shows that addiction is a cognitive disorder entailing maladaptive behavior and caused by long lasting neuroadaptations in the neurocircuitry of motivated behavior, and that addicts need to be treated clinically like any other neurological disorder (Kalivas and O'Brien, 2008). Drug addiction is often described as a cognitive disorder that is characterized by maladaptive decision-making and dysfunctional motivational circuits (Koob and Le Moal, 2001; Kalivas and Volkow, 2005; Schoenbaum et al., 2006). Addicts lack the necessary behavioral flexibility required to implement their stated desire to abstain from drug seeking, thereby limiting the efficacy of competing behaviors and extinction therapy in reducing drug-seeking and relapse. In addition to losing interest in obtaining natural rewards, addicts engage in compulsive drug seeking despite their conscious insights into the adverse outcomes of their decision/behavior (Kalivas and O'Brien, 2008).

2. Addiction is a Neurological Disorder

Addiction can be perceived as a “valuation disorder” where representations associated with the abused drug are overvalued, while other reward representations are undervalued (Montague, 2008). Drugs of abuse usurp the circuitry for regular motivated behavior and the mechanisms that are usually responsible for learning adaptive behaviors. This will modify the “conventional” neural representations of rewards and goal directed behaviors in the brain by highly overvaluing the abused drug. Therefore, from the evolutionary and neurobiological perspective of the addicted brain, procuring the drug is now regarded as an adaptive behavior that is more important than reproduction or even food. Thus, addicts technically make the “right” decision by choosing to seek the highly valued drugs. The problem however, is the pathologically high value associated with the drugs of abuse, resulting in behavioral inflexibility and loss of inhibitory control. Procuring drugs becomes the one and only “right” decision to make despite its negative consequences.

It is believed that by virtue of the endogenous functions of dopamine in the brain, recurrent surges in dopamine levels caused by repeated exposures to drugs (Di Chiara and Imperato, 1988; Kuczenski et al., 1991) induce synaptic plasticity and potentiate specific circuits in the brain encoding drug seeking (Chen et al., 2010). That is, while the rewarding effects of drugs like cocaine stem from the associated massive increase in dopamine levels throughout the reward circuitry, the compulsive drug-seeking behavior results from long-lasting neuroadaptations in the glutamatergic brain circuitry regulating motivated behaviors (Kalivas and Volkow, 2005; Graybiel, 2008; Kalivas, 2009). This circuitry was mapped and these neuroadaptations were identified using the animal models of drug seeking.

### B. Metabotropic Glutamate Receptor

Glutamate is the major excitatory neurotransmitter in the mammalian central nervous system (CNS). Glutamate produces its effects on central neurons by binding to and thereby activating cell surface receptors. These receptors have been divided into two major classes, the ionotropic and metabotropic glutamate receptors, based on the structural features of the receptor proteins, the means by which the receptors transduce signals into the cell, and pharmacological profiles.

The metabotropic glutamate receptors (mGluRs) are G protein-coupled receptors that activate a variety of intracellular second messenger systems following the binding of glutamate. Activation of mGluRs in intact mammalian neurons elicits one or more of the following responses: activation of phospholipase C; increases in phosphoinositide (PI) hydrolysis; intracellular calcium release; activation of phospholipase D; activation or inhibition of adenyl cyclase; increases or decreases in the formation of cyclic adenosine monophosphate (cAMP); activation of guanylyl cyclase; increases in the formation of cyclic guanosine monophosphate (cGMP); activation of phospholipase A₂; increases in arachidonic acid release; and increases or decreases in the activity of voltage- and ligand-gated ion channels. Schoepp et al., Trends Pharmacol. Sci. 14:13 (1993), Schoepp, Neurochem. Int. 24:439 (1994), Pin et al., Neuropharmacology 34:1 (1995), Bordi and Ugolini, Prog. Neurobiol. 59:55 (1999).

Metabotropic glutamate receptors have been implicated in a number of normal processes in the mammalian CNS. Activation of mGluRs has been shown to be required for induction of hippocampal long-term potentiation and cerebellar long-term depression, Bashir et al., Nature 363:347 (1993), Bortolotto et al., Nature 368:740 (1994), Aiba et al., Cell 79:365 (1994), Aiba et al., Cell 79:377 (1994). A role for mGluR activation in nociception and analgesia also has been demonstrated, Meller et al., Neuroreport 4: 879 (1993), Bordi and Ugolini, Brain Res. 871:223 (1999). In addition, mGluR activation has been suggested to play a modulatory role in a variety of other normal processes including synaptic transmission, neuronal development, apoptotic neuronal death, synaptic plasticity, spatial learning, olfactory memory, central control of cardiac activity, waking, motor control and control of the vestibulo-ocular reflex. Nakanishi, Neuron 13: 1031 (1994), Pin et al., Neuropharmacology 34:1, Knopfel et al., J. Med. Chem. 38:1417 (1995).

Molecular cloning has identified eight distinct mGluR subtypes, termed mGluR1 through mGluR8. Nakanishi, Neuron 13:1031 (1994), Pin et al., Neuropharmacology 34:1 (1995), Knopfel et al., J. Med. Chem. 38:1417 (1995). Further receptor diversity occurs via expression of alternatively spliced forms of certain mGluR subtypes. Pin et al., PNAS 89:10331 (1992), Minakami et al., BBRC 199:1136 (1994), Joly et al., J. Neurosci. 15:3970 (1995).

Metabotropic glutamate receptor subtypes may be subdivided into three groups, Group I, Group II, and Group III mGluRs, based on amino acid sequence homology, the second messenger systems utilized by the receptors, and by their pharmacological characteristics. mGluR1 and mGluR5 belong to group I, mGluR2 and mGluR3 belong to group II and mGluR4, mGluR6, mGluR7 and mGluR8 belong to group III. Alternatively spliced variants of these receptors are present as well. The binding of agonists to Group I receptors results in the activation of phospholipase C and the subsequent mobilization of intracellular calcium.

1. Group I mGluR

Group I metabotropic glutamate receptors and mGluR5 in particular, have been suggested to play roles in a variety of pathophysiological processes and disorders affecting the CNS. These include stroke, head trauma, anoxic and ischemic injuries, hypoglycemia, epilepsy, neurodegenerative disorders such as Alzheimer's disease and pain. Schoepp et al., Trends Pharmacol. Sci. 14:13 (1993), Cunningham et al., Life Sci. 54:135 (1994), Hollman et al., Ann. Rev. Neurosci. 17:31 (1994), Pin et al., Neuropharmacology 34:1 (1995), Knopfel et al., J. Med. Chem. 38:1417 (1995), Spooren et al., Trends Pharmacol. Sci. 22:331 (2001), Gasparini et al. Curr. Opin. Pharmacol. 2:43 (2002), Neugebauer Pain 98:1 (2002). Much of the pathology in these conditions is thought to be due to excessive glutamate-induced excitation of CNS neurons. Because Group I mGluR5 appear to increase glutamate-mediated neuronal excitation via postsynaptic mechanisms and enhanced presynaptic glutamate release, their activation probably contributes to the pathology. Accordingly, selective modulators of Group I mGluR receptors could be therapeutically beneficial, specifically as neuroprotective agents, analgesics or anticonvulsants. Further, it has also been shown that mGluR5 modulators are useful for the treatment of addictions or cravings (for drugs, tobacco, alcohol, any appetizing macronutrients or non-essential food items).

2. Group II mGluR

Group II metabotropic glutamate receptors (mGluR2/3) are densely expressed in the mesocorticolimbic and corticostriatal circuits. mGluR2/3s presynaptically control neurotransmitter release to regulate both reward processing and drug seeking, in part through their capacity to control release of dopamine and glutamate respectively. In pre-clinical models, mGluR2/3 receptor agonists administered systemically or locally into certain brain structures reduce the rewarding value of commonly abused drugs and inhibit the reinstatement of drug seeking.

The mGluR2/3 receptor family includes 2 subtypes both coupled to Gi proteins; mGluR2 receptors are expressed outside the active zone on presynaptic axon terminals to negatively regulate neurotransmitter release, while mGluR3 receptors are localized pre- and postsynaptically as well as on glia with a less clear overall function, but including negative regulation of transmitter release (Ohishi et al., 1993a; Testa et al., 1998; Schoepp, 2001; Tamaru et al., 2001; Richards et al., 2005). mGluR2/3 receptors can be homosynaptic, regulating glutamate release, or heterosynaptic regulating release of dopamine and γ-aminobutyric acid (GABA) (Hu et al., 1999; Schoepp, 2001; Karasawa et al., 2006; Xi et al., 2010). Gi coupling of mGluR2/3 receptors controls release through different mechanisms including activation of presynaptic K⁺ channels, inhibition of presynaptic Ca²⁺ channels, or direct interference with vesicular release (Anwyl, 1999).

In the PFC, mGluR2/3 appear to be tonically activated by endogenous glutamate. Microdialysis studies reveal an increase in PFC glutamate levels upon infusion of a selective mGluR2/3 receptor antagonist (LY341495) (Melendez et al., 2005; Xie and Steketee, 2008). However, perfusion of a selective agonist ((2R,4R)-4-aminopyrrolidine-2,4-dycarboxylate (APDC)) was without effect, suggesting the presence of ceiling-like glutamatergic tone on mGluR2/3 (Melendez et al., 2005). In addition, infusion of the antagonist LY341495 in the prefrontal cortex increased glutamate levels in subcortical regions of the reward circuitry including the nucleus accumbens and VTA (Xie and Steketee, 2008). This is possibly due to reduced inhibition resulting in facilitated excitatory output from the PFC.

In the nucleus accumbens, data indicate the presence of endogenous glutamatergic tone on mGluR2/3 receptors regulating both glutamate and dopamine levels. Electrophysiological recordings from accumbens slices reveal presynaptic autoregulation of glutamate release by mGluR2/3 receptors. Bath application of the selective agonists (S)-4-carboxy-3-hydroxyphenylglycine ((1S,3S)-ACPD) and (2S,1′S,2′S)-2-(2′-carboxycyclopropyl)glycine (L-CCG1) increased paired pulse ratios and reduced miniature excitatory post synaptic currents (mEPSC) frequency without affecting their amplitude, pointing to a presynaptic mode of action (Manzoni et al., 1997). In addition, in vivo microdialysis studies reveal glutamatergic tone on mGluR2/3 receptors as indicated by increased glutamate release upon selective antagonist LY143495 perfusion into the accumbens, while the agonist (APDC) reduced extracellular glutamate levels (Xi et al., 2002). mGluR2/3s regulate synaptic release in addition to glutamate efflux through Na⁺ independent cystine-glutamate antiporter through Ca²⁺ and protein kinase A (PKA) dependent cellular mechanisms (Xi et al., 2002).

Dopamine release in the accumbens is also controlled by mGluR2/3. Intra-accumbens infusion of direct (LY354740; (2S,1′R,2′R,3′R)-2-(2,3-dicarboxycyclopropyl)glycine (DCG-4); LY379268) or indirect agonists [2-(phosphonomethyl)-pentanedioic acid (2-PMPA) inhibits of N-acetylaspartylglutamate (NAAG) peptidase, thereby increasing NAAG levels, an endogenous mGlu3 receptor agonist] reduce, while antagonists (MGS0039; α-methyl-4-phosphonophenylglycine (MPPG)) increase basal dopamine levels measured with microdialysis probes (Hu et al., 1999; Greenslade and Mitchell, 2004; Karasawa et al., 2006; Xi et al., 2010). While this regulation depends on activation of voltage-dependent Ca²⁺ channels (Hu et al., 1999) pointing to vesicular release, it is not clear if it is mediated directly via heterosynaptic mGluR2/3 receptors on dopaminergic terminals, especially since some studies failed to identify significant mGluR2/3 receptor mRNA levels in midbrain neurons projecting to the ventral striatum (Ohishi et al., 1993b). Another possibility is that mGluR2/3 receptors regulate glutamatergic terminals on accumbens medium spiny neurons, which in turn project onto dopaminergic cells in the ventral tegmental area (VTA) (Kalivas et al., 1993). Furthermore, mGluR2/3 receptors regulate glutamate release in VTA (Manzoni and Williams, 1999), hippocampus (Marco, 2004), bed nucleus of stria terminalis (BNST) (Grueter and Winder, 2005) and other regions within the motivational circuit (Poisik et al., 2005).

### C. Cystine-Glutamate Exchange (System XC-)

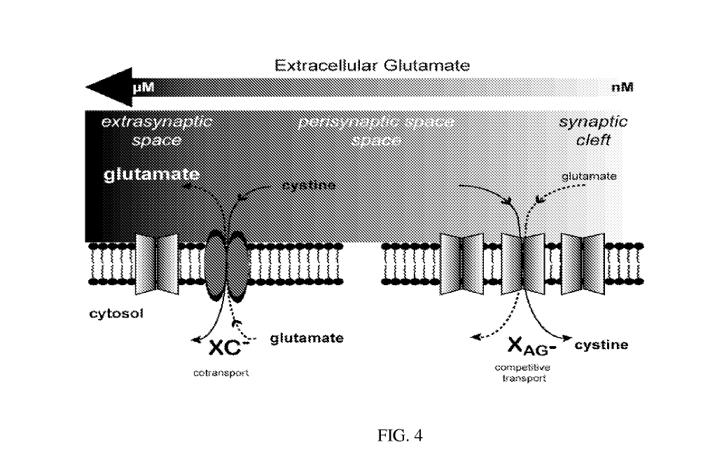

Extrasynaptic glutamate originates from non-vesicular release, most importantly from the cystine-glutamate exchanger (system XC⁻) (FIG. 4) (Westerink, 1995; Herrera-Marschitz et al., 1996; Timmerman and Westerink, 1997; Jabaudon et al., 1999; Baker et al., 2002; Xi et al., 2002; Melendez et al., 2005). Whereas inhibiting system X_{AG-} increases levels of extrasynaptic glutamate as measured with microdialysis, simply by blocking glutamate clearance, blocking system XC⁻ in the ventral striatum was shown to reduce extracellular glutamate levels by more than 60% (Baker et al., 2002; Xi et al., 2002). It was also found that knockdown of system XC⁻ in drosophilia reduces extracellular glutamate levels measured in larval hemolymph by 50% (Augustin et al., 2007). Furthermore, adding cystine to brain slices increases extracellular glutamate levels (Warr et al., 1999; Cavelier and Attwell, 2005; Moran et al., 2005).

System XC⁻ exchanges intracellular glutamate for extracellular cystine at a 1:1 ratio, which is rapidly reduced intracellularly into cysteine, the rate-limiting factor in glutathione biosynthesis (Bannai and Kitamura, 1980; Bannai, 1986; McBean, 2002; La Bella et al., 2007). System XC⁻ is a heterodimer composed of a heavy chain surface glycoprotein (4F2) and specific active subunit (xCT, 502 amino acids, 12 transmembrane domains), linked by a disulfide bridge (Sato et al., 1999; Deves and Boyd, 2000; Lewerenz et al., 2006).

XC⁻ is essential for neuronal survival (Shih et al., 2006). That is, by transporting cystine into astrocytes, system XC⁻ helps drive glutathione synthesis, which is then transported to the extracellular space to provide reduced thiol groups and prevent oxidative damage in neurons and brain tissue (Shih et al., 2006). XC⁻ is highly expressed in adult and to a lesser extent in fetal mammalian brain including striatum, cortex, hippocampus, cerebellum, and CSF-brain barrier (Sato et al., 1999; Sato et al., 2002; Burdo et al., 2006; Shih et al., 2006; La Bella et al., 2007). While it is usually expressed in glia and neurons (Sagara et al., 1993; Tang and Kalivas, 2003; Burdo et al., 2006; La Bella et al., 2007), system XC⁻ seems to be functional in glia but not in neurons (Sagara et al., 1993; Pow, 2001). The xCT gene is regulated by the transcriptional regulatory element “Antioxidant Response Element (ARE)” in its promoter region (Sasaki et al., 2002; Lo et al., 2008). Transcription factors Nrf-2 (Nuclear factor erythroid 2-related factor-2) and ATF4 (activating transcription factor-4) upregulate xCT expression (Lee and Johnson, 2004; Mann et al., 2007; Lewerenz and Maher, 2009), while c-Maf and Bach I transcription factors negatively regulate ARE-mediated gene expression (Dhakshinamoorthy and Jaiswal, 2002; Dhakshinamoorthy et al., 2005). In addition, activity of cystine-glutamate exchanger is dynamically regulated by dopamine-1 like receptors through protein kinase A (PKA) signaling cascade (Madayag et al., 2009), in addition to other pro-oxidant molecules (Bannai, 1984; Bannai et al., 1989; Bannai et al., 1991; Sato et al., 1995). This dopaminergic regulation of xCT could possibly underlie the reduced function and protein levels of xCT observed after exposure to cocaine, hence leading to reduced extrasynaptic glutamate levels and impaired glutamate homeostasis (Baker et al., 2003; Madayag et al., 2007; Kau et al., 2008; Kalivas, 2009; Knackstedt et al., 2010). Therefore, system XC⁻ emerged as potential therapeutic target in cocaine addiction, and several potential activators have been tested, one of which is N-acetylcysteine.

### D. N-acetylcysteine

N-acetylcysteine (NAC) (C₅H₉NO₃S, molecular weight 163.2) is currently used clinically as an antioxidant for the treatment of several disorders (Millea, 2009). It acts as prodrug for cystine and cysteine, the rate-limiting factors in the synthesis of the major endogenous antioxidant glutathione (Dringen and Hamprecht, 1999; Griffith, 1999; Sadowska et al., 2007). NAC is approved as an antidote for acetaminophen overdose (Prescott et al., 1977; Smilkstein et al., 1988), as a mucolytic agent for bronchopulmonary disorders with viscous secretions like cystic fibrosis (Grandjean et al., 2000a; Grandjean et al., 2000b), as a treatment for hyperhomocysteinemia, a major risk factor for vascular disease (Roes et al., 2002; Ventura et al., 2003; Scholze et al., 2004), and as an adjuvant to cancer chemotherapy to reduce liberated free radicals (Miller and Rumack, 1983). In addition several clinical trials and animal studies have shown a promising effect of NAC in several neurological disorders like Alzheimer's disease (Adair et al., 2001), mood disorders (Berk et al., 2008), and stroke (Knuckey et al., 1995). NAC side effects are minimal and mostly limited to gastrointestinal discomfort (Holdiness, 1991; Grandjean et al., 2000a).

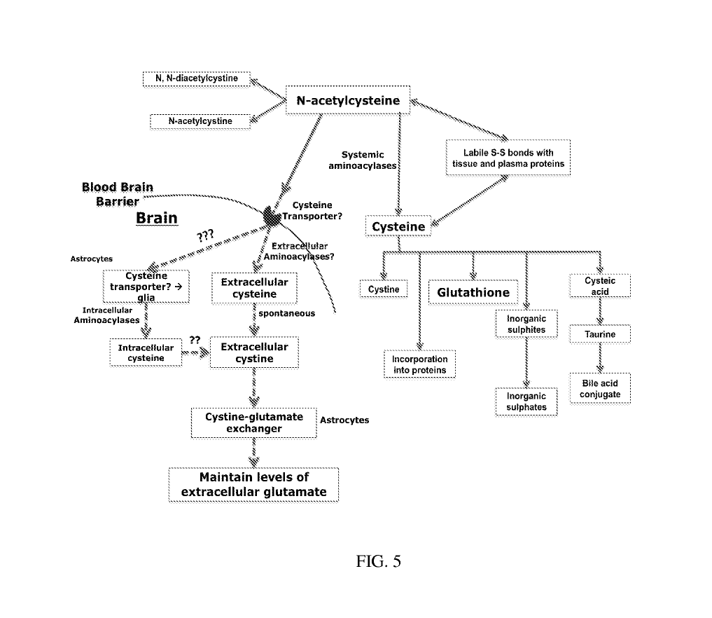

Oral NAC is extensively metabolized in the intestine and liver (Sjodin et al., 1989; Cotgreave, 1997) resulting in low oral bioavailability of about 4-10% with a peak plasma concentration reached 1-2 hours following its ingestion, and a half-life of 6.25 hours (Olsson et al., 1988; Holdiness, 1991). In humans, it was reported that a 400 or 600 mg oral dose of NAC results in a peak NAC plasma concentration of (10-17 μM) and 16 μM respectively within one hour of administration (Olsson et al., 1988; De Bernardi di Valserra et al., 1989; De Caro et al., 1989; Gabard and Mascher, 1991), while a 600 mg i.v. dose of NAC results in a peak concentration of 300 μM (De Caro et al., 1989). After absorption into plasma and tissues, NAC is present as the free form or as one of multiple metabolites (FIG. 5), but the free form is usually bound to proteins with a labile disulfide bond (De Caro et al., 1989).

NAC is commonly used as a prodrug for cysteine/cystine, which cannot be administered directly because of their instability. That is, cystine has very limited solubility and quickly precipitates in blood, and cysteine spontaneously oxidizes to cystine (Cho et al., 1984), making cystine and cysteine administration as treatments less than optimal (Yamauchi et al., 2002). On the other hand, NAC is more stable, more soluble, and less toxic (Yamauchi et al., 2002; Aoyama et al., 2006) and can be administered orally, nasally, or intravenously without any noticeable side effects (Brown et al., 2004; Millea, 2009). NAC is commonly cleaved by aminoacylases.

1. N-acetylcysteine Blocks Relapse and Restores Glutamate Homeostasis

In animal models of drug addiction, NAC prevents relapse to drug seeking (Baker et al., 2003; Moran et al., 2005; Madayag et al., 2007; Kau et al., 2008; Zhou and Kalivas, 2008). Acute administration of NAC reverses cocaine-induced neuroadaptations related to glutamate homeostasis and synaptic transmission in NAcore. By providing cystine and driving the blunted XC transporter (Kau et al., 2008), NAC normalizes extracellular glutamate levels in the accumbens after chronic cocaine (Baker et al., 2003; Madayag et al., 2007), and reduces synaptic glutamate release and excitatory drive from the PFC after cue or drug exposure (Baker et al., 2003; Madayag et al., 2007; Zhou and Kalivas, 2008), thereby preventing reinstatement of drug seeking.

In addition, chronic NAC treatment during cocaine self-administration or heroin extinction elicits enduring protection against relapse for several weeks after the last NAC injection (Madayag et al., 2007; Kau et al., 2008; Zhou and Kalivas, 2008), and this prolonged NAC effect on behavior is paralleled by reversal of molecular neuroadaptations associated with drawal from chronic cocaine; these include normalization of XC⁻ function and extracellular glutamate levels as well as inhibition of increased synaptic glutamate release after a drug prime (Madayag et al., 2007; Kau et al., 2008). NAC treatment during SA also reduces extinction responding after chronic cocaine (Kau et al., 2008) indicating a facilitation of extinction learning. Besides, NAC administered for 2 weeks during extinction from heroin self-administration reduces extinction responding in NAC treated rats and prevents reinstatement up to one month after the last NAC injection (Zhou and Kalivas, 2008). Moreover, NAC has been shown to reduce compulsive gambling behavior (Grant et al., 2007), desire for cocaine use (LaRowe et al., 2007), number of cigarettes smoked (Knackstedt et al., 2009), and marijuana abuse in humans (Gray et al., 2010).

### E. Compositions

Disclosed herein are compositions comprising a mGluR modulator and procysteine drug. The compositions can be a formulation comprising both the mGluR modulator and procysteine drug. The formulation can also comprise other pharmaceutically acceptable carriers.

In some forms of the disclosed compositions, the mGluR modulator can have the structure:

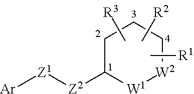

wherein the six membered ring defined by W¹, W² and carbon atoms 1, 2, 3 and 4, can be aromatic or non-aromatic, and further wherein any two neighboring atoms of this six membered ring may be singly or doubly bonded to one another;

Z¹ and Z² are either carbon or nitrogen, further wherein Z¹ and Z² can be singly, doubly, or triply bonded to one another and wherein Z² and carbon atom 1 can be singly or doubly bonded to one another, provided that the bond between Z¹ and Z² is not triple when Z₁ and Z₂ are nitrogen, further provided that the bond between Z¹ and Z² is single when the bond between Z² and carbon atom 1 is double;

Ar is substituted or unsubstituted aryl or substituted or unsubstituted heteroaryl;

one of either W¹ and W² is nitrogen and the other is carbon;

R¹, R², and R³ are independently hydrogen, hydroxy, amino, cyano, halo, nitro, mercapto, or a heteroatom-substituted or heteroatom-unsubstituted alkyl, alkenyl, alkynyl, aryl, aralkyl, acyl, alkoxy, alkylamino, or =0; or pharmaceutically acceptable salts, hydrates, tautomers, acetals, ketals, hemiacetals, hemiketals, or optical isomers thereof.

In some forms, Z₁ and Z₂ can be both carbon triply bonded to each other.

In some forms, Z₁ and Z₂ can be both nitrogen doubly bonded to each other.

In some forms, Ar can be phenyl, substituted phenyl, thiazole or substituted thiazole.

In some forms, W¹ can be nitrogen and W² can be carbon. In some forms, W² can be nitrogen and W¹ can be carbon.

In some forms, R¹, R², and R³ can be independently hydrogen, amino, alkyl, alkenyl or halo.

In some forms of the disclosed compositions, the mGluR modulator can be phenazopyridine; SIB 1893; SIB 1757; 2-methyl-6-(phenylethynyl)-pyridine (MPEP); NSC41777; 6-methyl-3-phenyldiazenylpyridin-2-amine; 2,6-Diamino-3-(4-iodophenylazo)pyridine; phenyldiazenylpyridin-2-amine; 3-(4-chlorophenyl)diazenylpyridine-2,6-diamine; 3-(2-chlorophenyl)diazenylpyridine-2,6-diamine; 3-[(2-methyl-1,3-thiazol-4-yl)ethynyl]pyridine (MTEP); 1-(3-chlorophenyl)-3-(3-methyl-5-oxo-4H-imidazol-2-yl)urea;

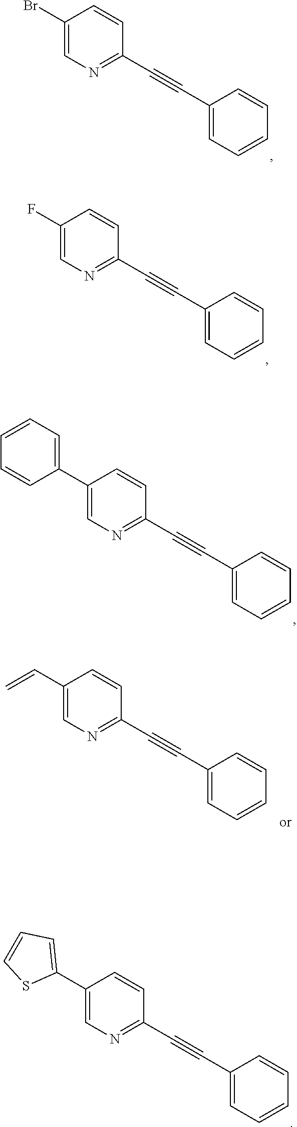

a physiologically acceptable salt thereof; or any mixture thereof.

In some forms of the disclosed compositions, the procysteine drug can be NAC, DiNAC; N-acetylcysteine L-lysine; Carbocisteine; glutathione; S-nitroso-N-acetylcysteine; S-nitrosothiol-N-acetylcysteine; S-allyl-cysteine; S-alkyl-cysteine; N-acetyl-S-farnesyl-cysteine; N-acetyl-L-arginine-NAC; N-acetyl-L-lysine-NAC; N-acetyl-L-histidine-NAC; N-acetyl-L-ornithine-NAC; thioester of NAC with salicylic acid; 2′4′-difluoro-4-hydroxy-(1,1′-diphenyl)-3-carboxylic derivatives of NAC; S-allymercapto-NAC (ASSNaC); N,N-diacetyl-L-cystine; N—S-diacyl-cysteine; N-acetylcysteine conjugate of phenethyl isothiocyanate (PEITC-NAC); S-carboxylmethyl-L-cysteine; derivatives of reacting a reactive derivative of p-isobutylphenylpropionic acid and NAC (e.g., an amide); paraisobutyl NAC; L-2-oxothiazolidine-4-carboxylic acid and a combination thereof. In some forms the procysteine drug have the structure:

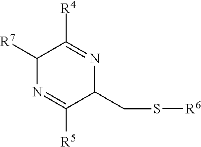

wherein: R4 and R5 can be independently selected from OH, ═O, or a branched or straight chain C1 to C5 alkoxyl group, with the caveat that when ═O is selected the nitrogen atom adjacent the carbonyl group thusly formed bears a H and a single bond joins the adjacent nitrogen to said carbonyl group;

R6 can be H, a branched or straight chain C1 to C5 alkyl, a nitrobenzenesulfonyl, a trityl, an aryl thio, an aryl, an alkylthio, an acyl, a benzoyl, a thio acyl, a thio benzoyl, or a benzyl group;

R7 can be selected from the side chain groups of the natural L-amino acids cys, gly, phe, pro, val, ser, arg, asp, asn, glu, gin, ala, his, ile, leu, lys, met, thr, trp, tyr, or D-isomers thereof, with the caveat that when R4 is the side chain group of the natural L-amino acid gly, R1 and R2 are not both selected to be ═O; or a cystine dimer of said prodrug having the structure:

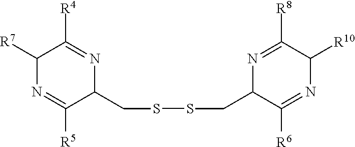

wherein: R4, R5, R8 and R9 can independently be selected from OH, ═O, or a branched or straight chain C1 to C5 alkoxyl group, with the caveat that when ═O is selected the nitrogen atom adjacent the carbonyl group thusly formed bears a H and a single bond joins the adjacent nitrogen to said carbonyl group; and

R7 and R10 can be independently selected from the side chain groups of the natural L-amino acids cys, gly, phe, pro, val, ser, arg, asp, asn, glu, gin, ala, his, ile, leu, lys, met, thr, trp, tyr, or D-isomers thereof, with the caveat that when R7 and R10 are both the side chain group of the natural L-amino acid gly, R4, R⁵, R⁸ and R⁹ shall not all be selected to be ═O.

In some forms the cysteine drug can have the structure:

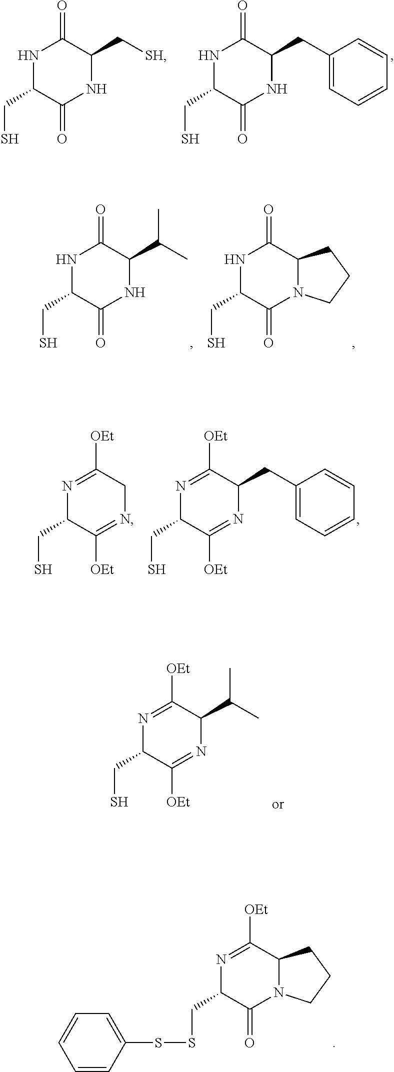

In some forms the cysteine drug is in the form of a cysteine dimer can have the structure:

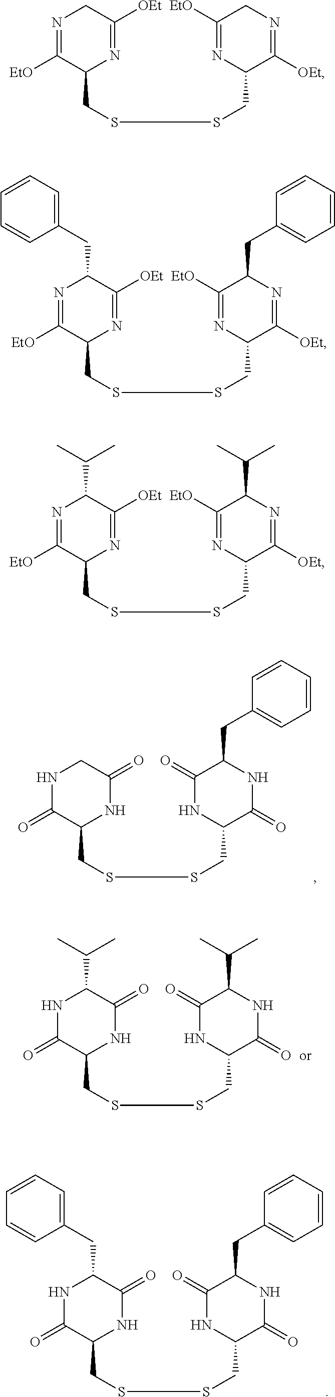

In some forms the cysteine drug is in the form of a cysteine dimer and R⁷ and R¹⁰ can be identical.

In some forms the cysteine drug is in the form of a cystine dimer and R⁷ and R¹⁰ can be non-identical.

In some forms the cysteine drug or cystine dimer thereof can include at least one R⁷ and R¹⁰ group that can be a cys, said cys is further protected by a branched or straight chain C₁ to C₅ alkyl, a nitrobenzenesulfonyl, a trityl, an aryl thio, an aryl, an alkylthio, an acyl, a benzoyl, a thio acyl, a thio benzoyl, or a benzyl group.

In some forms the cysteine drug can have the structure:

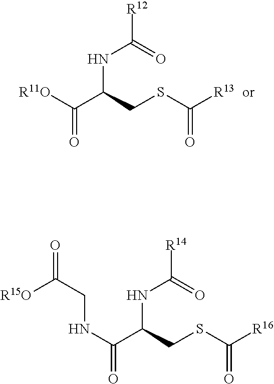

or a cystine dimer of the cysteine drug that can have the structure:

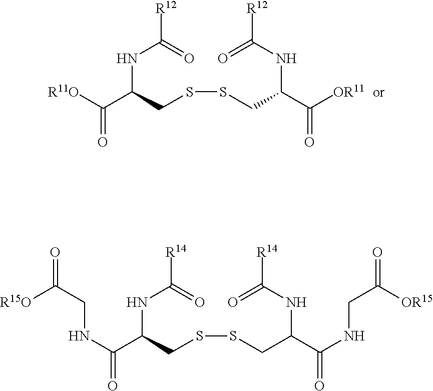

wherein R¹¹ through R¹⁶ are independently selected from a branched or straight chain C₁ to C₅ alkyl, a phenyl, or a benzyl group.

In some forms the cysteine drug can have the structure:

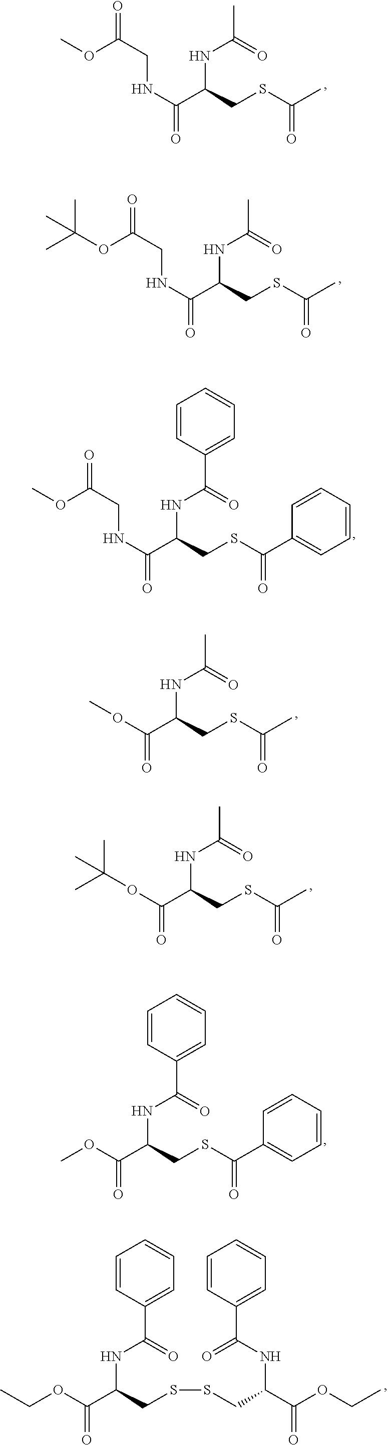

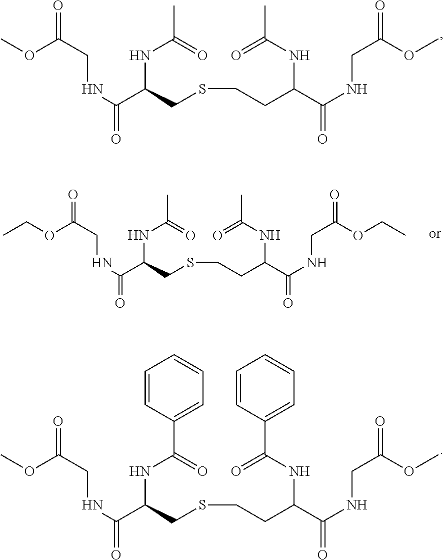

In some forms, the composition can reduce drug use. Drug seeking and relapse can also be reduced by the disclosed compositions.

In some forms, the mGluR modulator and procysteine drug can be each at subthreshold levels. In some forms, the mGluR modulator and procysteine drug can be each at therapeutic levels. In some forms, the mGluR modulator can be at subthreshold levels and the procysteine drug can be at therapeutic levels. In some forms, the mGluR modulator can be at therapeutic levels and the procysteine drug can be at subthreshold levels. The levels of each can vary. A variety of formulations can be possible.

In some forms of the disclosed compositions, side effects can be decreased compared to therapeutic levels of either compound alone. For example, a composition comprising subthreshold levels of a mGluR modulator and a procysteine drug can have decreased side effects compared to a composition comprising therapeutic levels of each. A decrease in side effects can result in the absence of side effects or can be the presence of the same side effects but with less frequency or less severity.

In some forms, the mGluR modulator can be a mGluR5 modulator. In some forms the mGluR5 modulator can be a negative mGluR5 modulator. In some forms, the negative mGluR5 modulator can be a negative allosteric mGluR5 modulator. In some forms, the mGluR5 modulator can be 3-((2-Methyl-4-thiazolyl)ethynyl)pyridine (MTEP). In some forms, the mGluR5 modulator can be 2-methyl-6-(phenylethynyl)pyridine (MPEP). Another example of a mGluR modulator is Fenobam or 1-(3-chlorophenyl)-3-(3-methyl-5-oxo-4H-imidazol-2-yl)urea.

In some forms, the procysteine drug can be NAC. Another example of a procysteine drug is OTZ (L-2-oxothiazolidine-4-carboxylic acid).

In some forms of the disclosed compositions, the drug addiction can be cocaine addiction. Other examples of drug addictions can be, but are not limited to, a nicotine, marijuana, amphetamine, sedative, opiate, barbituate, or hallucinogen addictions.

### F. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for treating subjects with addiction, the kit comprising, for example, a mGluR modulator and a procysteine drug. The kits also can contain a vehicle for administering the compositions. Reagents and other materials, such as those described herein, can also be included, alone or in combination.

### G. Mixtures

Disclosed are mixtures formed by performing or preparing to perform the disclosed method. For example, disclosed are mixtures of mGluR modulators, procysteine drugs and pharmaceutically acceptable carriers. Also disclosed are mixtures of the ligand and the composition, such as mGluR5 and the mGluR modulator.

Whenever the method involves mixing or bringing into contact compositions or components or reagents, performing the method creates a number of different mixtures. For example, if the method includes 3 mixing steps, after each one of these steps a unique mixture is formed if the steps are performed separately. In addition, a mixture is formed at the completion of all of the steps regardless of how the steps were performed. The present disclosure contemplates these mixtures, obtained by the performance of the disclosed methods as well as mixtures containing any disclosed reagent, composition, or component, for example, disclosed herein.

### H. Systems

Disclosed are systems useful for performing, or aiding in the performance of, the disclosed method. Systems generally comprise combinations of articles of manufacture such as structures, machines, devices, and the like, and compositions, compounds, materials, and the like. Such combinations that are disclosed or that are apparent from the disclosure are contemplated. In certain embodiments, the compositions can be considered a system, along with things such as pharmaceutically acceptable carriers.

### I. Data Structures and Computer Control

Disclosed are data structures used in, generated by, or generated from, the disclosed method. Data structures generally are any form of data, information, and/or objects collected, organized, stored, and/or embodied in a composition or medium. A compositions formulation stored in electronic form, such as in RAM or on a storage disk, is a type of data structure.

The disclosed method, or any part thereof or preparation therefor, can be controlled, managed, or otherwise assisted by computer control. Such computer control can be accomplished by a computer controlled process or method, can use and/or generate data structures, and can use a computer program. Such computer control, computer controlled processes, data structures, and computer programs are contemplated and should be understood to be disclosed herein. Furthermore, the data obtained from using the compositions can be collected, stored, and manipulated on computer systems.

### J. Uses

The disclosed methods and compositions are applicable to numerous areas including, but not limited to, treating subjects with current or prior addiction. The compositions can also be used as research tools. Other uses are disclosed, apparent from the disclosure, and/or will be understood by those in the art.

### K. Methods

Disclosed herein are methods of treating subjects comprising, administering a metabotropic glutamate receptor (mGluR) modulator and a procysteine drug, wherein the subject has had a prior addiction. The prior addiction can be a drug addiction. The prior addiction can be, but is not limited to, a drug, gambling, sex or alcohol addiction. The drug addiction can be a legal or illegal drug. The drug addiction can be a cocaine addiction. Other examples of drug addictions can be, but are not limited to, a nicotine, marijuana, amphetamine, sedative, opiate, barbituate, or hallucinogen addictions.

In some forms of the disclosed methods, the combination of these compounds can reduce drug use. For example, administering to subjects the combination of a mGluR modulator and a procysteine drug can reduce current drug use or future drug use. A reduction in future drug use can be a reduction in drug addiction relapse. Reducing drug use, drug seeking or relapse can be a decrease in the amount of drug use or drug seeking or an increase in the time between the last drug use and the next drug use. For example, a subject administered a mGluR modulator and a procysteine drug can either use less drug or can increase the time between drug uses. Any reduction in drug use, drug seeking or relapse can be acceptable.

In some forms of the disclosed methods, the mGluR can be mGluR5. Modulating mGluR5 by altering, blocking or antagonizing the receptor can result in decreased drug use, drug seeking or relapse. There are several mGluR5 modulators. In some forms of the disclosed methods, the mGluR or mGluR5 modulator can be a negative modulator. In some forms, the negative modulator can be a negative allosteric modulator. The mGluR5 modulator can be 2-methyl-6-(phenylethynyl)pyridine (MPEP). The mGluR5 modulator can be 3-((2-Methyl-4-thiazolyl)ethynyl)pyridine (MTEP). Another example of a mGluR modulator is Fenobam or 1-(3-chlorophenyl)-3-(3-methyl-5-oxo-4H-imidazol-2-yl)urea.

In some forms of the disclosed methods, the procysteine drug can be N-acetylcysteine (NAC). Another example of a procysteine drug is OTZ (L-2-oxothiazolidine-4-carboxylic acid). The NAC can activate mGluR2/3. NAC activation of mGluR2/3 can occur by NACs increase of the cystine-glutamate exchange which results in higher levels of extracellular glutamate (Glu). Extracellular Glu can then bind mGluR2/3 which inhibits or reduces the release of Glu into the synapse. The lack of or decrease in synaptic Glu reduces drug seeking.

In some forms of the disclosed methods, the mGluR modulator can be administered at subthreshold levels. In some forms, the mGluR modulator is administered at subthreshold levels and the procysteine drug is administered at therapeutic levels. In some forms, the procysteine drug can be administered at subthreshold levels. In some forms the procysteine drug can be administered at subthreshold levels and the mGluR can be administered at therapeutic levels. And in some forms, the mGluR modulator and procysteine drug are both administered at subthreshold levels. The mGluR modulator and procysteine drug can have a synergistic effect. For example, administering either the mGluR modulator or the procysteine drug alone at subthreshold levels can result in no reduction of drug use, drug seeking or relapse. However, the combination of the two can have a synergistic effect and reduce drug use, drug seeking or relapse in subjects with prior addiction even though they are administered at subthreshold levels. The synergistic effect can result in a reduction in drug use, drug seeking or relapse equal to or better than administering either drug alone at a therapeutic dose. Administering subthreshold levels can be advantageous because it can allow for a decrease or absence of toxicity or side effects that may result from therapeutic levels of the mGluR modulator or procysteine drug alone.

In some forms of the disclosed methods, the mGluR modulator and procysteine drug each can be administered at therapeutic levels. The mGluR modulator can be administered at therapeutic levels. In some forms, the procysteine drug can be administered at therapeutic levels. For example, administering either the mGluR modulator or the procysteine drug at therapeutic levels can result in a reduction of drug use, drug seeking or relapse in subjects with prior addiction. The combination of the two can have a synergistic effect and reduce drug use, drug seeking or relapse in subjects with prior addiction better than either the mGluR modulator or the procysteine drug could alone.

In some forms of the disclosed methods, the mGluR modulator and procysteine drug can be administered simultaneously. Simultaneous administration can be the mGluR modulator and the procysteine drug in two separate formulations but administered at the same time. In some forms, the mGluR modulator and procysteine drug are administered in one composition or formulation. Simultaneous administration does not have to be administration at the exact same time but instead can be the administration of the mGluR modulator and the procysteine drug within 1, 2, 3, 4, 5, 6, 7, 8, 9, 10, 15, 20, 25 or 30 minutes of each other. Furthermore, the mGluR modulator or the procysteine drug can be given first.

In some forms of the disclosed methods, the mGluR modulator and procysteine drug are administered consecutively. Consecutive administration can be the administration of the mGluR modulator and procysteine drug at least 30 minutes apart. They can be administered 1, 2, 3, 4, 5, 10, 15, 20 hours apart. They can be administered 1, 2, 3, 4, 5, 6 or 7 days apart. They can be administered 1, 2, 3, 4, 5, 6, 7, 8, 9 or 10 weeks apart. The order in which they are administered can vary. The mGluR can be administered first or the procysteine drug can be administered first. In some forms, the procysteine drug can be administered chronically and the mGluR modulator administered acutely at varying times. In some forms of the disclosed methods, the mGluR modulator and procysteine drug can be administered before the subject encounters a drug cue. Administering before a subject encounters a drug cue can be any time up until the subject encounters the drug cue. Thus, it can be hours, days, weeks, months or years before the subject encounters a drug cue. In some forms, the mGluR modulator and procysteine drug can be administered in the presence of a drug cue. In some forms, the mGluR modulator and procysteine drug can be administered after the subject encounters the drug cue. Administering the mGluR modulator and procysteine drug after the subject encounters the drug cue can be 5, 10, 15, 20, 25, 30, 45 or 60 minutes after encountering the drug cue. In some forms it can be administered 1, 2, 3, 4, 5, 6, 7, 8, 9, 10, 15, 20, 24 hours after encountering the drug cue. In some forms it can be administered 1, 2, 3, 4, 5, 6 or 7 days after encountering the drug cue. In some forms, the procysteine drug can be chronically administered before the subject encounters the drug cue and the mGluR modulator can be administered either before the subject encounters the drug cue or after the subject encounters the drug cue.

In some forms of the disclosed methods, the mGluR modulator and procysteine drug can be administered intraperitoneally. The mGluR modulator and procysteine drug can be administered in a variety of way. Administration can be, but is not limited to, oral, intravenous, intraperitoneal, intramuscular, subcutaneous, intranasal, intradermal, as well as, by transdermal delivery, or even by gastrointestinal delivery. In some forms, administration can be a direct administration to the brain.

In some forms of the disclosed methods, side effects are decreased in subjects administered both the mGluR modulator and procysteine drug compared to subjects administered either of them alone. Therapeutic levels of a mGluR modulator and a procysteine drug can lead to unwanted side effects such as nauseau, headaches, and delirium. In order to reduce or get rid of the side effects, the levels of mGluR modulator and procysteine drug can be decreased.

Disclosed herein are methods of inhibiting drug seeking comprising identifying a subject at risk for drug use and administering a mGluR modulator and a procysteine drug to the subject. Addiction can result in an addict actively seeking out their drug of choice (drug seeking). Inhibiting drug seeking can be a treatment for drug addicts. Also disclosed herein are methods of preventing drug use in a subject comprising identifying the subject as being at risk for drug use and administering a mGluR modulator and a procysteine drug to the subject. As with drug seeking, preventing drug use can be an effective treatment for drug addicts. Also disclosed herein are methods of decreasing glutamate release in the neuronal synapse and glutamate binding to mGluR5 comprising administering a mGluR modulator and a procysteine drug to a subject at risk for drug use. Glutamate release into the synapse can result in drug use, drug seeking or relapse. In some forms, preventing glutamate release into the synapse can be achieved with a procysteine drug which increases extracellular glutamate that binds to mGluR2/3 and reduces the release of glutamate into the synapse. The binding of extracellular glutamate to mGluR5 can be inhibited by a mGluR modulator, particularly a negative allosteric modulator, which antagonizes the mGluR5 altering the receptor conformation and preventing glutamate from binding.

In some forms of the disclosed methods, the drug use can be cocaine use. The drug use can be, but is not limited to, use of cocaine, marijuana, opiates, benzodiazepines, amphetamines, nicotine, ethanol, sedatives, opiates, barbituates, or hallucinogens.

Also described herein is a method of treating subjects comprising, administering a metabotropic glutamate receptor (mGluR) modulator and a procysteine drug, wherein the subject has had a prior addiction, wherein the combination of these compounds can reduce relapse of addiction, wherein the mGluR modulator have the structure of:

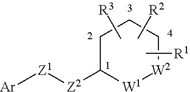

wherein the six membered ring defined by W¹, W² and carbon atoms 1, 2, 3 and 4, can be aromatic or non-aromatic, and further wherein any two neighboring atoms of this six membered ring can be singly or doubly bonded to one another;

Z¹ and Z² can either be carbon or nitrogen, further wherein Z¹ and Z² can be singly, doubly, or triply bonded to one another and wherein Z² and carbon atom 1 can be singly or doubly bonded to one another, provided that the bond between Z¹ and Z² cannot be triple when Z₁ and Z₂ are nitrogen, further provided that the bond between Z¹ and Z² can be single when the bond between Z² and carbon atom 1 can be double;

Ar can be substituted or unsubstituted aryl or substituted or unsubstituted heteroaryl;

one of either W¹ and W² can be nitrogen and the other can be carbon;

R¹, R², and R³ can independently be hydrogen, hydroxy, amino, cyano, halo, nitro, mercapto, or a heteroatom-substituted or heteroatom-unsubstituted alkyl, alkenyl, alkynyl, aryl, aralkyl, acyl, alkoxy, alkylamino, or =0; or pharmaceutically acceptable salts, hydrates, tautomers, acetals, ketals, hemiacetals, hemiketals, or optical isomers thereof.

In one embodiment Z₁ and Z₂ can both be carbon triply bonded to each other.

In another embodiment Z₁ and Z₂ can both be nitrogen doubly bonded to each other.

In another embodiment Ar can be phenyl, substituted phenyl, thiazole or substituted thiazole.

In another embodiment W¹ can be nitrogen and W² can be carbon.

In another embodiment W² can be nitrogen and W¹ can be carbon.

In another embodiment R¹, R², and R³ can independently be hydrogen, amino, alkyl, alkenyl or halo.

In another embodiment the mGluR modulator can be: phenazopyridine; SIB 1893; SIB 1757; 2-methyl-6-(phenylethynyl)-pyridine (MPEP); NSC41777; 6-methyl-3-phenyldiazenylpyridin-2-amine; 2,6-Diamino-3-(4-iodophenylazo)pyridine; phenyldiazenylpyridin-2-amine; 3-(4-chlorophenyl)diazenylpyridine-2,6-diamine; 3-(2-chlorophenyl)diazenylpyridine-2,6-diamine; 3-[(2-methyl-1,3-thiazol-4-yl)ethynyl]pyridine (MTEP); 1-(3-chlorophenyl)-3-(3-methyl-5-oxo-4H-imidazol-2-yl)urea;

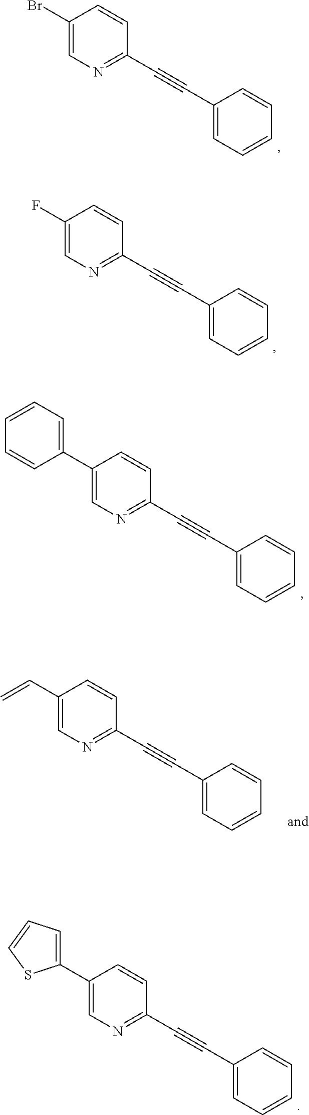

a physiologically acceptable salt thereof; or any mixture thereof.

In another embodiment the procysteine drug is NAC, DiNAC; N-acetylcysteine L-lysine; Carbocisteine; glutathione; S-nitroso-N-acetylcysteine; S-nitrosothiol-N-acetylcysteine; S-allyl-cysteine; S-alkyl-cysteine; N-acetyl-S-farnesyl-cysteine; N-acetyl-L-arginine-NAC; N-acetyl-L-lysine-NAC; N-acetyl-L-histidine-NAC; N-acetyl-L-ornithine-NAC; thioester of NAC with salicylic acid; 2′4′-difluoro-4-hydroxy-(1,1′-diphenyl)-3-carboxylic derivatives of NAC; S-allymercapto-NAC (ASSNaC); N,N-diacetyl-L-cystine; N—S-diacyl-cysteine; N-acetylcysteine conjugate of phenethyl isothiocyanate (PEITC-NAC); S-carboxylmethyl-L-cysteine; derivatives of reacting a reactive derivative of p-isobutylphenylpropionic acid and NAC (e.g., an amide); paraisobutyl NAC; and a combination thereof

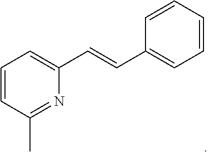

SIB 1893 has the structure:

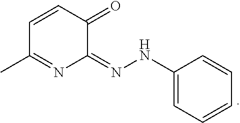

SIB 1757 has the structure:

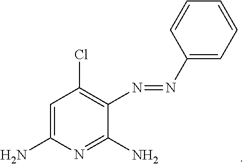

NSC41777 has the strucutre:

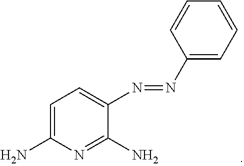

Phenazopyridine has the structure:

In some forms of the methods the procysteine drug have the structure:

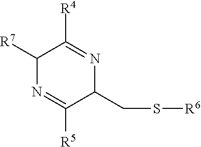

wherein: R⁴ and R⁵ can be independently selected from OH, ═O, or a branched or straight chain C₁ to C₅ alkoxyl group, with the caveat that when ═O is selected the nitrogen atom adjacent the carbonyl group thusly formed bears a H and a single bond joins the adjacent nitrogen to said carbonyl group;

R⁶ can be H, a branched or straight chain C₁ to C₅ alkyl, a nitrobenzenesulfonyl, a trityl, an aryl thio, an aryl, an alkylthio, an acyl, a benzoyl, a thio acyl, a thio benzoyl, or a benzyl group;

R⁷ can be selected from the side chain groups of the natural L-amino acids cys, gly, phe, pro, val, ser, arg, asp, asn, glu, gin, ala, his, ile, leu, lys, met, thr, trp, tyr, or D-isomers thereof, with the caveat that when R⁴ is the side chain group of the natural L-amino acid gly, R¹ and R² are not both selected to be ═O; or a cystine dimer of said prodrug having the structure:

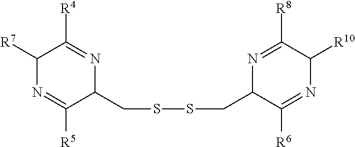

wherein: R⁴, R⁵, R⁸ and R⁹ can independently be selected from OH, ═O, or a branched or straight chain C₁ to C₅ alkoxyl group, with the caveat that when ═O is selected the nitrogen atom adjacent the carbonyl group thusly formed bears a H and a single bond joins the adjacent nitrogen to said carbonyl group; and

R⁷ and R¹⁰ can be independently selected from the side chain groups of the natural L-amino acids cys, gly, phe, pro, val, ser, arg, asp, asn, glu, gin, ala, his, ile, leu, lys, met, thr, trp, tyr, or D-isomers thereof, with the caveat that when R⁷ and R¹⁰ are both

the side chain group of the natural L-amino acid gly, R⁴, R⁵, R⁸ and R⁹ shall not all be selected to be ═O.

In some forms of the methods the cysteine drug can have the structure:

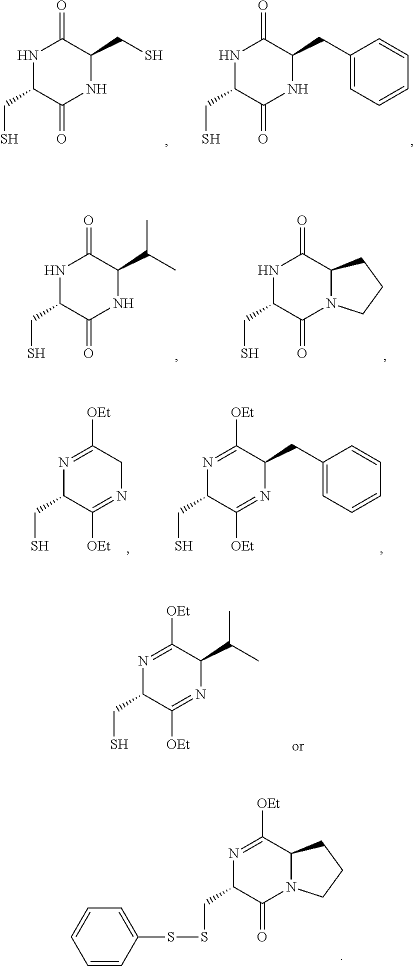

In some forms of the methods the cysteine drug is in the form of a cysteine dimer can have the structure:

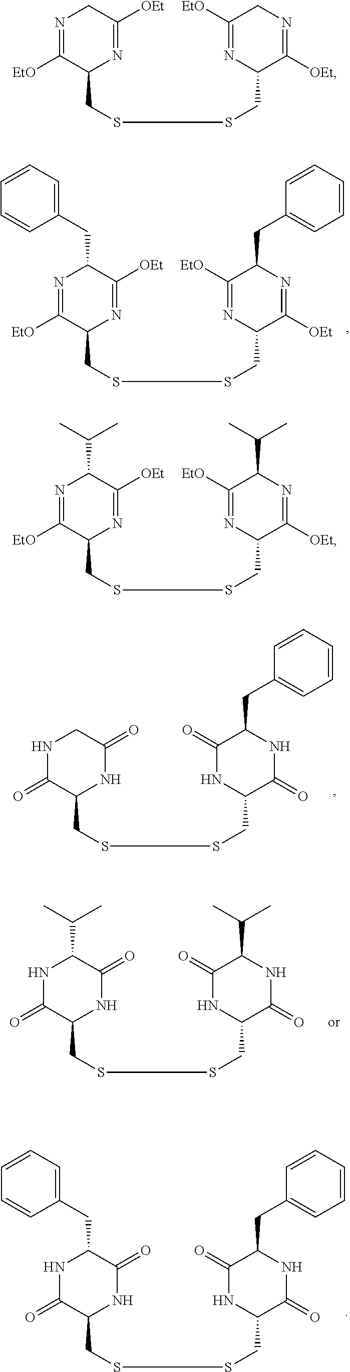

In some forms of the methods the cysteine drug is in the form of a cysteine dimer and R⁷ and R¹⁰ can be identical.

In some forms of the methods cysteine drug is in the form of a cystine dimer and R⁷ and R¹⁰ can be non-identical.

In some forms of the methods the cysteine drug or cystine dimer thereof can include at least one R⁷ and R¹⁰ group that can be a cys, said cys is further protected by a branched or straight chain C₁ to C₅ alkyl, a nitrobenzenesulfonyl, a trityl, an aryl thio, an aryl, an alkylthio, an acyl, a benzoyl, a thio acyl, a thio benzoyl, or a benzyl group.

In some forms of the methods the cysteine drug can have the structure:

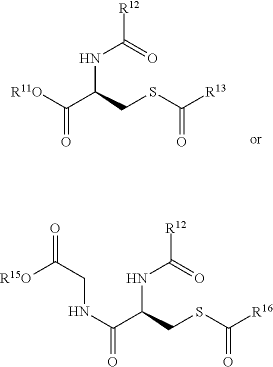

or a cystine dimer of the cysteine drug that can have the structure:

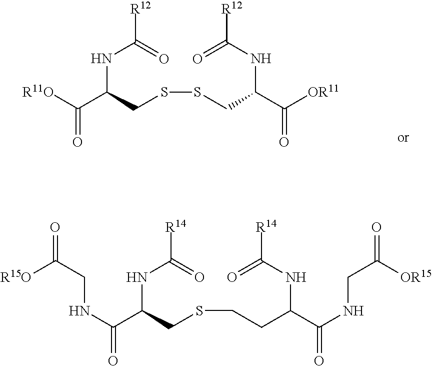

wherein R¹¹ through R¹⁶ are independently selected from a branched or straight chain C₁ to C₅ alkyl, a phenyl, or a benzyl group.

In some forms of the methods the cysteine drug can have the structure:

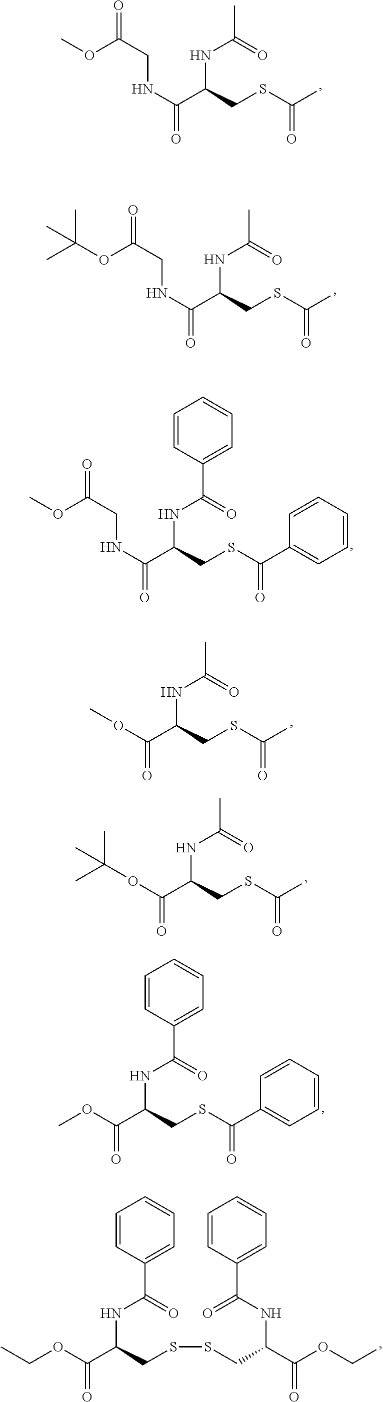

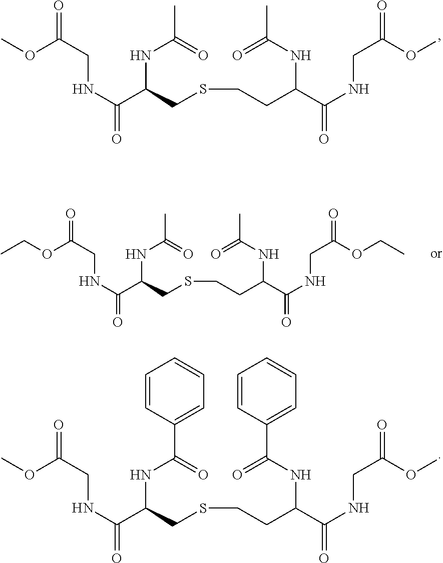

L. Delivery

The route of delivery of the mGluR modulators or procysteine drugs disclosed herein can be determined by the particular disorder. They may be delivered orally, intravenously, intraperitoneally, intramuscularly, subcutaneously, intranasally, and intradermally, as well as, by transdermal delivery (e.g., with a lipid-soluble carrier in a skin patch placed on skin), or even by gastrointestinal delivery (e.g., with a capsule or tablet).

Furthermore, the mGluR modulators and procystein drugs, in certain aspects, can be delivered directly to the brain or certain regions of the brain (e.g. nucleus accumbens) to activate or inhibit receptors at specific brain sites producing the desirable effect without inhibiting or activating receptors at other brain sites, thus avoiding undesirable side-effects or actions that may counteract the beneficial therapeutic action mediated by the former site (s). The dosage will be sufficient to provide an effective amount of an mGluR modulator and procysteine drug either singly or in combination. Some variation in dosage will necessarily occur depending upon the condition of the patient being treated, and the physician will, in any event, determine the appropriate dose for the individual patient. The dose will depend, among other things, on the body weight, physiology, and chosen administration regimen.

The mGluR modulators and procysteine drugs disclosed herein can be administered alone or in combination with pharmaceutical acceptable carriers, in either single or multiple doses.

Suitable pharmaceutical carriers include inert solid diluents or fillers, sterile aqueous solutions, and various nontoxic organic solvents. The pharmaceutical compositions formed by combining one or more modulator with the pharmaceutically acceptable carrier are then readily administered in a variety of dosage forms such as tablets, lozenges, syrups, injectable solutions, and the like. These pharmaceutical carriers can, if desired, contain additional ingredients such as flavorings, binders, excipients, and the like. Thus, for purposes of oral administration, tablets containing various excipients such as sodium citrate, calcium carbonate, and calcium phosphate are employed along with various disintegrants such as starch, and preferably potato or tapioca starch, alginic acid, and certain complex silicates, together with binding agents such as polyvinylpyrolidone, sucrose, gelatin, and acacia. Additionally, lubricating agents, such as magnesium stearate, sodium lauryl sulfate, and talc are often useful for tableting purposes. Solid compositions of a similar type may also be employed as fillers in salt and hard-filled gelatin capsules. Preferred materials for this purpose include lactose or milk sugar and high molecular weight polyethylene glycols.

When aqueous suspensions of elixirs are desired for oral administration, the compositions may be combined with various sweetening or flavoring agents, colored matter or dyes, and if desired, emulsifying or suspending agents, together with diluents such as water, ethanol, propylene glycol, glycerin, and combinations thereof. For parenteral administration, solutions of preparation in sesame or peanut oil or in aqueous polypropylene glycol are employed, as well as sterile aqueous saline solutions of the corresponding water soluble pharmaceutical acceptable metal salts previously described. Such an aqueous solution should be suitably buffered if necessary and the liquid diluent first rendered isotonic with sufficient saline or glucose. These particular aqueous solutions are especially suitable for intravenous, intramuscular, subcutaneous, and intraperitoneal injection. The sterile aqueous media employed are all readily obtainable by standard techniques well known to those skilled in the art.

### M. Definitions

1. A

As used in the specification and the appended claims, the singular forms “a,” “an” and “the” or like terms include plural referents unless the context clearly dictates otherwise. Thus, for example, reference to “a pharmaceutical carrier” includes mixtures of two or more such carriers, and the like.

2. Abbreviations

Abbreviations, which are well known to one of ordinary skill in the art, may be used (e.g., “h” or “hr” for hour or hours, “g” or “gm” for gram(s), “mL” for milliliters, and “rt” for room temperature, “nm” for nanometers, “M” for molar, and like abbreviations).

3. About

About modifying, for example, the quantity of an ingredient in a composition, concentrations, volumes, process temperature, process time, yields, flow rates, pressures, and like values, and ranges thereof, employed in describing the embodiments of the disclosure, refers to variation in the numerical quantity that can occur, for example, through typical measuring and handling procedures used for making compounds, compositions, concentrates or use formulations; through inadvertent error in these procedures; through differences in the manufacture, source, or purity of starting materials or ingredients used to carry out the methods; and like considerations. The term “about” also encompasses amounts that differ due to aging of a composition or formulation with a particular initial concentration or mixture, and amounts that differ due to mixing or processing a composition or formulation with a particular initial concentration or mixture. Whether modified by the term “about” the claims appended hereto include equivalents to these quantities.

4. Addiction

Addiction implies that an individual has first “learned” how to be dependent before being addicted. Addiction can be a substance dependence which is diagnosed based on presence of three or more of the following criteria: tolerance, withdrawal, large amounts over a long period, unsuccessful efforts to cut down, time spent in obtaining the substance replaces social, occupational or recreational activities, and continued use despite adverse consequences.

5. Combinations

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a cell is disclosed and discussed and a number of modifications that can be made to a number of molecules including the cell are discussed, each and every combination and permutation of the cell and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

6. Comprise

Throughout the description and claims of this specification, the word “comprise” and variations of the word, such as “comprising” and “comprises,” means “including but not limited to,” and is not intended to exclude, for example, other additives, components, integers or steps.

7. Decrease

A “decrease” can refer to any change that results in a smaller amount of a composition, compound or action, such as drug use. Thus, a “decrease” can refer to a reduction in levels, function, or activity. Also for example, a decrease can be a change in the amount of drug use such that the drug use can be less than previously observed. Another example can be a decrease in the side effects in subjects administered a combination composition compared to side effects in subjects administered each compositions alone.

8. Drug Cue

The phrase “drug cue” can refer to anything that is associated with the initial drug addiction and thus may stimulate a relapse. A drug cue can be, but is not limited to, a person, a specific location, music, food, alcohol, or any substance. For example, if every time an addict was with a particular person they used cocaine, then that person could be a drug cue. The addict would associate drug use with that person and thus, interaction with that person could cause relapse, drug use or drug seeking.

9. Drug Seeking

The phrase “drug seeking” can refer to the activity of looking for drugs. Drug seeking refers to behavior aimed at obtaining a drug or substance, even in the face of negative health and social consequences. Drug seeking is often uncontrollable and compulsive. For example, a cocaine addict will actively seek cocaine in order to fulfill their addiction needs.

10. mGluR Modulator

An mGluR modulator is a substance which alters or modulates the normal signal or response through the mGluR. Thus, the modulator may be, for example, a chemical modulator, a pharmacokinetic modulator, an modulator by receptor block, a non-competitive modulator or a physiological modulator.

11. mGluR5 Modulator

An mGluR5 modulator is a substance which alters or modulates the normal signal or response through the mGluR5. Thus, the modulator may be, for example, a chemical modulator, a pharmacokinetic modulator, an modulator by receptor block, a non-competitive modulator or a physiological modulator.

12. Modulator

A modulator or like terms is a molecule that controls the activity of a cellular target, such as GluR5. A modulator can increase or decrease the activity of the target.

13. A Negative Modulator

A negative modulator or like terms refers to a modulator which decreases the activity of the cellular target.

14. A Positive Modulator

A positive modulator or like terms refers to a modulator which increases the activity of the cellular target.

15. Negative Allosteric Modulator

The phrase “negative allosteric modulator” can mean a negative modulator that decreases the activity of the target through an allosteric mechanism. For example, a negative allosteric modulator can antagonize a receptor by producing a conformational change in the receptor that lowers the receptor's ability to bind to its natural ligand. For example, MPEP can be a negative allosteric modulator of mGluR in that it causes a conformational change in mGluR that lowers mGluR's ability to bind glutamate. In some instances, a negative allosteric modulator can be called an antagonist. For example, MPEP can be an mGluR5 antagonist.

16. Or

The word “or” or like terms as used herein means any one member of a particular list and also includes any combination of members of that list.

17. Prevent

By “prevent” or other forms of prevent means to stop a particular characteristic or condition. Prevent does not require comparison to a control as it is typically more absolute than, for example, reduce or inhibit. As used herein, something could be reduced but not inhibited or prevented, but something that is reduced could also be inhibited or prevented. It is understood that where reduce, inhibit or prevent are used, unless specifically indicated otherwise, the use of the other two words is also expressly disclosed. Thus, if inhibits phosphorylation is disclosed, then reduces and prevents phosphorylation are also disclosed.

18. Publications

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

19. Ranges

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

20. Reduce

By “reduce” or other forms of reduce means lowering of an event or characteristic. It is understood that this is typically in relation to some standard or expected value, in other words it is relative, but that it is not always necessary for the standard or relative value to be referred to. For example, “reduces phosphorylation” means lowering the amount of phosphorylation that takes place relative to a standard or a control.

21. Subthreshold Levels

The term “subthreshold levels” can mean a dose that is ineffective on its own for treating a disease or disorder. A Subthreshold level is a level below the threshold level. Subthreshold levels can be doses of compounds that do not elicit a measurable response that can be elicited by higher doses. For example, subthreshold levels of the mGluR5 modulator, MPEP, are ineffective at reducing drug use, drug seeking or relapse in subjects with prior addiction but higher, or therapeutic levels, of MPEP can be effective at reducing drug use, drug seeking or relapse.

22. Synergistic Effect

The phrase “synergistic effect” refers to the effect seen by the combination of one or more things. A synergistic effect is an effect or result of two or more things which is greater than the sum of each thing individually. For example, the administration of mGluR modulator alone or procysteine drug alone can result in a reduction in drug use, drug seeking or relapse but administration of both of them can result in a greater reduction in drug use, drug seeking or relapse greater. In another example, subthreshold levels of a mGluR modulator and a procysteine drug individual can have no effect on drug use but administration of subthreshold levels of both, in combination, can have a synergistic effect resulting in a decrease in drug use.

23. Threshold Level

A threshold level is the level at which a compound has a particular effect. For a particular molecule, a threshold level can be determined by titrating the amount of the molecule, until an effect is seen, and the amount at which the effect is seen is the threshold level.

24. Therapeutic Levels

The term “therapeutic levels” can mean a dose that is effective on its own to treat a disease or disorder. For example, therapeutic levels of the mGluR5 modulator, MPEP, are effective at reducing drug use, drug seeking or relapse in subjects with prior addiction. Therapeutic levels, although effective at treating a disease or disorder, can have side effects.

25. Subject at Risk for Relapse Behavior

A “subject at risk for relapse behavior” can be identified by family, friends or a professional. A subject at risk for relapse behavior can be a subject where the relapse behavior would be drug use, a subject at risk for drug use. Those at risk for relapse behavior can be, but are not limited to, individuals who have used drugs in the past or are currently using drugs. In one instance, a subject who uses marijuana can be considered at risk for using cocaine. A subject at risk for relapse behavior can be a subject having had a prior addiction.

26. Chemistry

i. Alkyl

The term “alkyl” as used herein is a branched or unbranched saturated hydrocarbon moiety. “Unbranched” or “Branched” alkyls comprise a non-cyclic, saturated, straight or branched chain hydrocarbon moiety having from 1 to 24 carbons, 1 to 20 carbons, 1 to 15 carbons, 1 to 12 carbons, 1 to 8 carbons, 1 to 6 carbons, or 1 to 4 carbon atoms. It is understood that the term “alkyl” also encompass straight or branched chain hydrocarbon moiety having 1, 2, 3, 4, 5, 6, 7, 8, 9, 10, 11, 12, 13, 14, 15, 16, 17, 18, 19, 20, 21, 22, 23, or 24 carbon atoms. Examples of such alkyl radicals include methyl, ethyl, propyl, butyl, pentyl, hexyl, heptyl, octyl, n-propyl, iso-propyl, butyl, n-butyl, sec-butyl, t-butyl, amyl, t-amyl, n-pentyl and the like. Lower alkyls comprise a noncyclic, saturated, straight or branched chain hydrocarbon residue having from 1 to 4 carbon atoms, i.e., C₁-C₄ alkyl.

Moreover, the term “alkyl” as used throughout the specification and claims is intended to include both “unsubstituted alkyls” and “substituted alkyls”, the later denotes an alkyl radical analogous to the above definition that is further substituted with one, two, or more additional organic or inorganic substituent groups. Suitable substituent groups include but are not limited to H, alkyl, alkenyl, alkynyl, hydroxyl, cycloalkyl, heterocyclyl, amino, mono-substituted amino, di-substituted amino, unsubstituted or substituted amido, carbonyl, halogen, sulfhydryl, sulfonyl, sulfonato, sulfamoyl, sulfonamide, azido, acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy, haloalkoxy, heteroaryl, substituted heteroaryl, aryl or substituted aryl. It will be understood by those skilled in the art that an “alkoxy” can be a substituted of a carbonyl substituted “alkyl” forming an ester. When more than one substituent group is present then they can be the same or different. The organic substituent moieties can comprise from 1 to 12 carbon atoms, or from 1 to 6 carbon atoms, or from 1 to 4 carbon atoms. It will be understood by those skilled in the art that the moieties substituted on the “alkyl” chain can themselves be substituted, as described above, if appropriate.

ii. Alkenyl

The term “alkenyl” as used herein is an alkyl residue as defined above that also comprises at least one carbon-carbon double bond in the backbone of the hydrocarbon chain. Examples include but are not limited to vinyl, allyl, 2-butenyl, 3-butenyl, 2-pentenyl, 3-pentenyl, 4-pentenyl, 2-hexenyl, 3-hexenyl, 4-hexenyl, 5-hexanyl, 2-heptenyl, 3-heptenyl, 4-heptenyl, 5-heptenyl, 6-heptenyl and the like. The term “alkenyl” includes dienes and trienes of straight and branch chains.

iii. Alkynyl

The term “alkynyl” as used herein is an alkyl residue as defined above that comprises at least one carbon-carbon triple bond in the backbone of the hydrocarbon chain. Examples include but are not limited ethynyl, 1-propynyl, 2-propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl, 5-hexynyl and the like. The term “alkynyl” includes di- and tri-ynes.

iv. Cycloalkyl

The term “cycloalkyl” as used herein is a saturated hydrocarbon structure wherein the structure is closed to form at least one ring. Cycloalkyls typically comprise a cyclic radical containing 3 to 8 ring carbons, such as cyclopropyl, cyclobutyl, cyclopentyl, cyclopenyl, cyclohexyl, cycloheptyl and the like. Cycloalkyl radicals can be multicyclic and can contain a total of 3 to 18 carbons, or preferably 4 to 12 carbons, or 5 to 8 carbons. Examples of multicyclic cycloalkyls include decahydronapthyl, adamantyl, and like radicals.

Moreover, the term “cycloalkyl” as used throughout the specification and claims is intended to include both “unsubstituted cycloalkyls” and “substituted cycloalkyls”, the later denotes an cycloalkyl radical analogous to the above definition that is further substituted with one, two, or more additional organic or inorganic substituent groups that can include but are not limited to hydroxyl, cycloalkyl, amino, mono-substituted amino, di-substituted amino, unsubstituted or substituted amido, carbonyl, halogen, sulfhydryl, sulfonyl, sulfonato, sulfamoyl, sulfonamide, azido, acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy, haloalkoxy, heteroaryl, substituted heteroaryl, aryl or substituted aryl. When the cycloalkyl is substituted with more than one substituent group, they can be the same or different. The organic substituent groups can comprise from 1 to 12 carbon atoms, or from 1 to 6 carbon atoms, or from 1 to 4 carbon atoms.

v. cycloalkenyl

The term “cycloalkenyl” as used herein is a cycloalkyl radical as defined above that comprises at least one carbon-carbon double bond. Examples include but are not limited to cyclopropenyl, 1-cyclobutenyl, 2-cyclobutenyl, 1-cyclopentenyl, 2-cyclopentenyl, 3-cyclopentenyl, 1-cyclohexyl, 2-cyclohexyl, 3-cyclohexyl and the like.

vi. Alkoxy

The term “alkoxy” as used herein is an alkyl residue, as defined above, bonded directly to an oxygen atom, which is then bonded to another moiety. Examples include methoxy, ethoxy, n-propoxy, iso-propoxy, n-butoxy, t-butoxy, iso-butoxy and the like

vii. Amino

The term “amino” as used herein is a moiety comprising a N radical substituted with zero, one or two organic substituent groups, which include but are not limited to alkyls, alkyls, cycloalkyls, aryls, or arylalkyls. If there are two substituent groups they can be different or the same. Examples of amino groups include, —NH₂, methylamino (—NH—CH₃); ethylamino (—NHCH₂CH₃), hydroxyethylamino (—NH—CH₂CH₂OH), dimethylamino, methylethylamino, diethylamino, and the like.

viii. Mono-Substituted Amino

The term “mono-substituted amino” as used herein is a moiety comprising an NH radical substituted with one organic substituent group, which include but are not limited to alkyls, substituted alkyls, cycloalkyls, aryls, or arylalkyls. Examples of mono-substituted amino groups include methylamino (—NH—CH₃); ethylamino (—NHCH₂CH₃), hydroxyethylamino (—NH—CH₂CH₂OH), and the like.

ix. Di-Substituted Amino

The term “di-substituted amino” as used herein is a moiety comprising a nitrogen atom substituted with two organic radicals that can be the same or different, which can be selected from but are not limited to aryl, substituted aryl, alkyl, substituted alkyl or arylalkyl, wherein the terms have the same definitions found throughout. Some examples include dimethylamino, methylethylamino, diethylamino and the like.

x. Azide

As used herein, the term “azide”, “azido” and their variants refer to any moiety or compound comprising the monovalent group —N₃ or the monovalent ion —N₃

xi. Haloalkyl

The term “haloalkyl” as used herein an alkyl residue as defined above, substituted with one or more halogens, preferably fluorine, such as a trifluoromethyl, pentafluoroethyl and the like.

xii. Haloalkoxy

The term “haloalkoxy” as used herein a haloalkyl residue as defined above that is directly attached to an oxygen to form trifluoromethoxy, pentafluoroethoxy and the like.

xiii. Acyl

The term “acyl” as used herein is a R—C(O)— residue having an R group containing 1 to 8 carbons. The term “acyl” encompass acyl halide, R—(O)-halogen. Examples include but are not limited to formyl, acetyl, propionyl, butanoyl, iso-butanoyl, pentanoyl, hexanoyl, heptanoyl, benzoyl and the like, and natural or un-natural amino acids.

xiv. Acyloxy

The term “acyloxy” as used herein is an acyl radical as defined above directly attached to an oxygen to form an R—C(O)O— residue. Examples include but are not limited to acetyloxy, propionyloxy, butanoyloxy, iso-butanoyloxy, benzoyloxy and the like.

xv. Aryl

The term “aryl” as used herein is a ring radical containing 6 to 18 carbons, or preferably 6 to 12 carbons, comprising at least one aromatic residue therein. Examples of such aryl radicals include phenyl, naphthyl, and ischroman radicals. Moreover, the term “aryl” as used throughout the specification and claims is intended to include both “unsubstituted alkyls” and “substituted alkyls”, the later denotes an aryl ring radical as defined above that is substituted with one or more, preferably 1, 2, or 3 organic or inorganic substituent groups, which include but are not limited to a halogen, alkyl, alkenyl, alkynyl, hydroxyl, cycloalkyl, amino, mono-substituted amino, di-substituted amino, unsubstituted or substituted amido, carbonyl, halogen, sulfhydryl, sulfonyl, sulfonato, sulfamoyl, sulfonamide, azido acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy or haloalkoxy, aryl, substituted aryl, heteroaryl, heterocyclic ring, ring wherein the terms are defined herein. The organic substituent groups can comprise from 1 to 12 carbon atoms, or from 1 to 6 carbon atoms, or from 1 to 4 carbon atoms. An aryl moiety with 1, 2, or 3 alkyl substituent groups can be referred to as “arylalkyl.” It will be understood by those skilled in the art that the moieties substituted on the “aryl” can themselves be substituted, as described above, if appropriate.

xvi. Heteroaryl

The term “heteroaryl” as used herein is an aryl ring radical as defined above, wherein at least one of the ring carbons, or preferably 1, 2, or 3 carbons of the aryl aromatic ring has been replaced with a heteroatom, which include but are not limited to nitrogen, oxygen, and sulfur atoms. Examples of heteroaryl residues include pyridyl, bipyridyl, furanyl, and thiofuranyl residues. Substituted “heteroaryl” residues can have one or more organic or inorganic substituent groups, or preferably 1, 2, or 3 such groups, as referred to herein-above for aryl groups, bound to the carbon atoms of the heteroaromatic rings. The organic substituent groups can comprise from 1 to 12 carbon atoms, or from 1 to 6 carbon atoms, or from 1 to 4 carbon atoms.

xvii. Heterocyclyl

The term “heterocyclyl” or “heterocyclic group” as used herein is a non-aromatic mono- or multi ring radical structure having 3 to 16 members, preferably 4 to 10 members, in which at least one ring structure include 1 to 4 heteroatoms (e.g. O, N, S, P, and the like). Heterocyclyl groups include, for example, pyrrolidine, oxolane, thiolane, imidazole, oxazole, piperidine, piperizine, morpholine, lactones, lactams, such as azetidiones, and pyrrolidiones, sultams, sultones, and the like. Moreover, the term “heterocyclyl” as used throughout the specification and claims is intended to include both “unsubstituted alkyls” and “substituted alkyls”, the later denotes an aryl ring radical as defined above that is substituted with one or more, preferably 1, 2, or 3 organic or inorganic substituent groups, which include but are not limited to a halogen, alkyl, alkenyl, alkynyl, hydroxyl, cycloalkyl, amino, mono-substituted amino, di-substituted amino, unsubstituted or substituted amido, carbonyl, halogen, sulfhydryl, sulfonyl, sulfonato, sulfamoyl, sulfonamide, azido acyloxy, nitro, cyano, carboxy, carboalkoxy, alkylcarboxamido, substituted alkylcarboxamido, dialkylcarboxamido, substituted dialkylcarboxamido, alkylsulfonyl, alkylsulfinyl, thioalkyl, thiohaloalkyl, alkoxy, substituted alkoxy or haloalkoxy, aryl, substituted aryl, heteroaryl, heterocyclic ring, ring wherein the terms are defined herein. The organic substituent groups can comprise from 1 to 12 carbon atoms, or from 1 to 6 carbon atoms, or from 1 to 4 carbon atoms. It will be understood by those skilled in the art that the moieties substituted on the “heterocyclyl” can themselves be substituted, as described above, if appropriate.

xviii. Halogen or Halo

The term “halo” or “halogen” refers to a fluoro, chloro, bromo or iodo group.

xix. Moiety

A “moiety” is part of a molecule (or compound, or analog, etc.). A “functional group” is a specific group of atoms in a molecule. A moiety can be a functional group or can include one or functional groups.

xx. Ester

The term “ester” as used herein is represented by the formula —C(O)OA, where A can be an alkyl, halogenated alkyl, alkenyl, alkynyl, aryl, heteroaryl, cycloalkyl, cycloalkenyl, heterocycloalkyl, or heterocycloalkenyl group described above.

xxi. Carbonate Group

The term “carbonate group” as used herein is represented by the formula —OC(O)OR, where R can be hydrogen, an alkyl, alkenyl, alkynyl, aryl, aralkyl, cycloalkyl, halogenated alkyl, or heterocycloalkyl group described above.

xxii. Keto Group

The term “keto group” as used herein is represented by the formula —C(O)R, where R is an alkyl, alkenyl, alkynyl, aryl, aralkyl, cycloalkyl, halogenated alkyl, or heterocycloalkyl group described above.

xxiii. Aldehyde

The term “aldehyde” as used herein is represented by the formula —C(O)H or —R—C(O)H, wherein R can be as defined above alkyl, alkenyl, alkoxy, aryl, heteroaryl, cycloalkyl, cycloalkenyl, heterocycloalkyl, or heterocycloalkenyl group described above.

xxiv. Carboxylic Acid

The term “carboxylic acid” as used herein is represented by the formula —C(O)OH.

xxv. Carbonyl Group

The term “carbonyl group” as used herein is represented by the formula C═O.

xxvi. Ether

The term “ether” as used herein is represented by the formula AOA¹, where A and A¹ can be, independently, an alkyl, halogenated alkyl, alkenyl, alkynyl, aryl, heteroaryl, cycloalkyl, cycloalkenyl, heterocycloalkyl, or heterocycloalkenyl group described above.

xxvii. Urethane

The term “urethane” as used herein is represented by the formula —OC(O)NRR′, where R and R′ can be, independently, hydrogen, an alkyl, alkenyl, alkynyl, aryl, aralkyl, cycloalkyl, halogenated alkyl, or heterocycloalkyl group described above.

xxviii. Silyl Group

The term “silyl group” as used herein is represented by the formula —SiRR′R″, where R, R′, and R″ can be, independently, hydrogen, an alkyl, alkenyl, alkynyl, aryl, aralkyl, cycloalkyl, halogenated alkyl, alkoxy, or heterocycloalkyl group described above.

xxix. Sulfo-Oxo Group

The term “sulfo-oxo group” as used herein is represented by the formulas —S(O)₂R, —OS(O)₂R, or, —OS(O)₂OR, where R can be hydrogen or as defined above an alkyl, alkenyl, alkynyl, aryl, aralkyl, cycloalkyl, halogenated alkyl, or heterocycloalkyl group described above.

### EXAMPLES

### A. Results

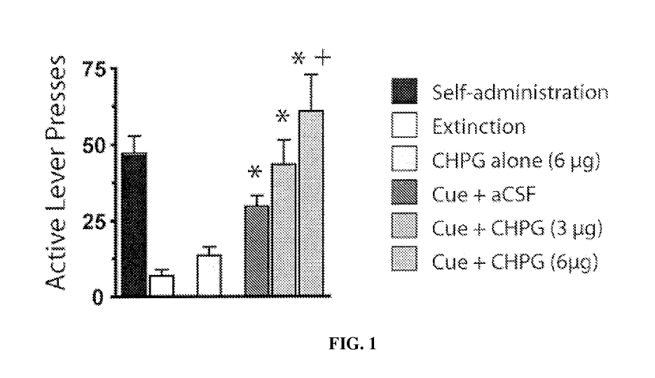

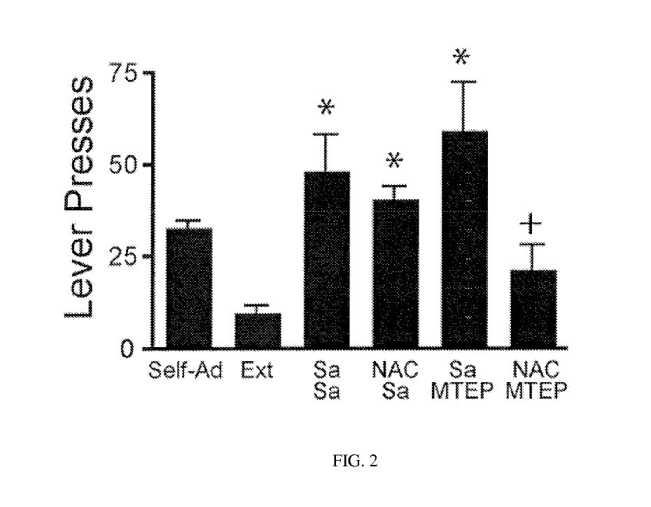

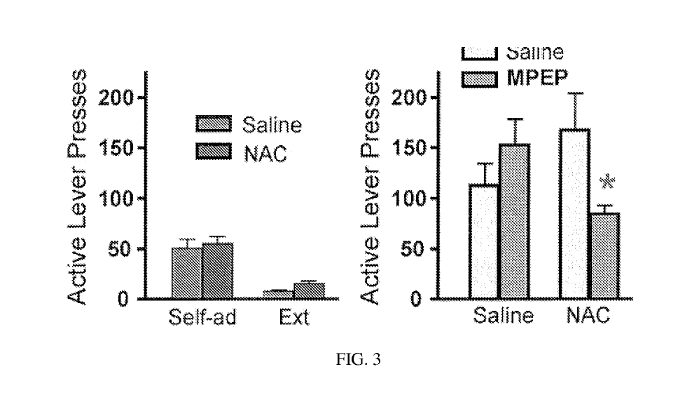

A number of previous studies have shown that mGluR5 modulators inhibit relpase in animal models (Backstrom et al., 2004; Backstrom et al., 2006; Palmatier et al., 2008; Olive, 2009) and recently it was found that increasing extreacellualr glutamate in the presence of an mGluR5 allosteric agonist will promote relapse (Moussawi et al., 2009). FIG. 1 extends this observation by showing that direct stimulation of mGluR5 in the nucleus accumbens (a brain region mediating relapse) increases relapse induced by cocaine cues. However, when given alone this agonist was ineffective at producing relapse. N-acetylcysteine (NAC) has also been shown previously to inhibit relapse to cocaine and heroin in animal models (Baker et al., 2003; Zhou et al., 2007). FIG. 2 shows that when subthreshold doses of mGluR5 modulator (MTEP) and NAC were given simultaneously a synergistic inhibition of relapse was produced. This is important given studies showing that in humans NAC reduces but does not abolish cocaine, nicotine and marijuana use. These findings indicate that a combination of NAC and mGluR5 modulator can provide full protection from relapse. Towards this end, the effects of chronic NAC followed by acute administration of mGluR5 modulator (MPEP) on relapse in the rat model were examined. FIG. 3 shows that MPEP reduced cocaine relapse in the chronic NAC animals, but was without effect in the chronic saline animals.

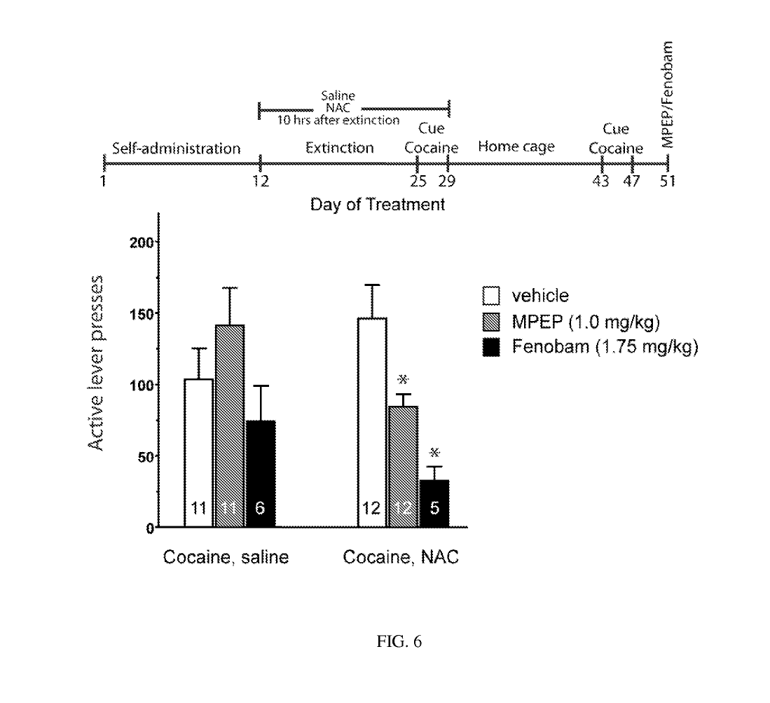

Based on the ability of NAC-driven glutamate to stimulate both mGluR2/3 and mGluR5 metabotropic receptor subtypes, greater stimulation of mGluR5 receptors can mask the effect of NAC on mGluR2/3 resulting in a lack of effect on drug seeking. Rats were injected with low doses of MPEP (1.0 mg/kg/ip) or Fenobam (1.75 mg/kg/ip) (FIG. 6), both systemically active and selective mGluR5 receptor antagonists (Carroll, 2008; Montana et al., 2009). Interestingly, both drugs significantly reduced lever pressing in NAC but not vehicle treated rats (Appendix, Figure A-10). These results indicate that when NAC is injected after extinction training sessions, blocking NAC-induced increased stimulation of mGluR5 receptors unmasks the NAC-induced mGluR2/3 stimulation and inhibits drug seeking akin to NAC administered 2 hours before extinction. Thus, a combination of NAC and mGluR5 antagonists are a great therapeutic strategy for addiction.

### B. Methods

i. Animal Housing and Surgery

All experiments were conducted in accordance with the US National Institutes of Health guidelines for the care and use of laboratory animals, and all procedures were approved by the Institutional Animal Care and Use Committee at the Medical University of South Carolina. One week after arrival, male Sprague-Dawley rats (250 g) were anesthetized with ketamine HCl (87.5 mg kg⁻¹ Ketaset, Fort Dodge Animal Health) and xylazine (5 mg kg⁻¹ Rompum, Bayer) and implanted with intravenous catheters. The catheters were flushed daily with cefazolin (0.2 ml of a 0.1 g/ml solution) and heparin (0.2 ml of 100 units) to prevent infection and maintain catheter patency. Rats were allowed to recover for a week before behavioral training (Baker et al. Nat. Neurosci 6:743-749, 2003).

ii. Self-Administration and Extinction Periods

Rats were trained to self-administer cocaine in a standard operant chamber with two retractable levers. The self-administration regimen consisted of 10d of self-administration at >10 infusions per session. Daily sessions lasted 2 hr, with an active lever press resulting in a cocaine infusion of 0.2 mg in 0.05 ml over 3 s, whereas inactive lever presses were of no consequence. Each infusion was followed by a 20 s timeout during which lever presses did not result in further cocaine infusion. Cocaine self-administering rats were often paired with yoked saline controls. Extinction procedures began 24 h after the rat met the acquisition criterion and lasted for at least 3 weeks. Lever presses were inconsequential throughout the extinction training

### REFERENCES

Backstrom P, Hyytia P (2006) Ionotropic and metabotropic glutamate receptor antagonism attenuates cue-induced cocaine seeking. Neuropsychopharmacology 31:778-786.
Backstrom P, Bachteler D, Koch S, Hyytia P, Spanagel R (2004) mGluR5 modulator MPEP reduces ethanol-seeking and relapse behavior. Neuropsychopharmacology 29:921-928.
Moussawi K, Pacchioni A, Moran M, Olive M F, Gass J T, Lavin A, Kalivas P W (2009) N-Acetylcysteine reverses cocaine-induced metaplasticity. Nat Neurosci 12:182-189.
Olive M F (2009) Metabotropic glutamate receptor ligands as potential therapeutics for addiction. Curr Drug Abuse Rev 2:83-989.
Palmatier M I, Liu X, Donny E C, Caggiula A R, Sved A F (2008) Metabotropic Glutamate 5 Receptor (mGluR5) Modulators Decrease Nicotine Seeking, But Do Not Affect the Reinforcement Enhancing Effects of Nicotine. Neuropsychopharmacology 33:2139-2147.
Adair J C, Knoefel J E, Morgan N (2001) Controlled trial of N-acetylcysteine for patients with probable Alzheimer's disease. Neurology 57:1515-1517.
Anwyl R (1999) Metabotropic glutamate receptors: electrophysiological properties and role in plasticity. Brain Res Brain Res Rev 29:83-120.
Aoyama K, Suh S W, Hamby A M, Liu J, Chan W Y, Chen Y, Swanson R A (2006) Neuronal glutathione deficiency and age-dependent neurodegeneration in the EAAC1 deficient mouse. Nat Neurosci 9:119-126.
Augustin H, Grosjean Y, Chen K, Sheng Q, Featherstone D E (2007) Nonvesicular release of glutamate by glial xCT transporters suppresses glutamate receptor clustering in vivo. J Neurosci 27:111-123.
Baker D A, Xi Z X, Shen H, Swanson C J, Kalivas P W (2002) The origin and neuronal function of in vivo nonsynaptic glutamate. J Neurosci 22:9134-9141.
Baker D A, McFarland K, Lake R W, Shen H, Tang X C, Toda S, Kalivas P W (2003) Neuroadaptations in cystine-glutamate exchange underlie cocaine relapse. Nat Neurosci 6:743-749.
Bannai S (1984) Induction of cystine and glutamate transport activity in human fibroblasts by diethyl maleate and other electrophilic agents. J Biol Chem 259:2435-2440.
Bannai S (1986) Exchange of cystine and glutamate across plasma membrane of human fibroblasts. J Biol Chem 261:2256-2263.
Bannai S, Kitamura E (1980) Transport interaction of L-cystine and L-glutamate in human diploid fibroblasts in culture. J Biol Chem 255:2372-2376.
Bannai S, Sato H, Ishii T, Sugita Y (1989) Induction of cystine transport activity in human fibroblasts by oxygen. J Biol Chem 264:18480-18484.
Bannai S, Sato H, Ishii T, Taketani S (1991) Enhancement of glutathione levels in mouse peritoneal macrophages by sodium arsenite, cadmium chloride and glucose/glucose oxidase. Biochim Biophys Acta 1092:175-179.
Berk M, Copolov D L, Dean O, Lu K, Jeavons S, Schapkaitz I, Anderson-Hunt M, Bush A I (2008) N-acetyl cysteine for depressive symptoms in bipolar disorder—a double-blind randomized placebo-controlled trial. Biol Psychiatry 64:468-475.
Booth R E, Watters J K, Chitwood D D (1993) HIV risk-related sex behaviors among injection drug users, crack smokers, and injection drug users who smoke crack. Am J Public Health 83:1144-1148.
Brown M, Bjorksten A, Medved I, McKenna M (2004) Pharmacokinetics of intravenous N-acetylcysteine in men at rest and during exercise. Eur J Clin Pharmacol 60:717-723.
Brust J C, Richter R W (1977) Stroke associated with cocaine abuse—? N Y State J Med 77:1473-1475.
Burdo J, Dargusch R, Schubert D (2006) Distribution of the cystine/glutamate antiporter system xc- in the brain, kidney, and duodenum. J Histochem Cytochem 54:549-557.
Carroll F I (2008) Antagonists at metabotropic glutamate receptor subtype 5: structure activity relationships and therapeutic potential for addiction. Ann N Y Acad Sci 1141:221-232.
Cavelier P, Attwell D (2005) Tonic release of glutamate by a DIDS-sensitive mechanism in rat hippocampal slices. J Physiol 564:397-410.
Chen B T, Hopf F W, Bonci A (2010) Synaptic plasticity in the mesolimbic system: therapeutic implications for substance abuse. Ann N Y Acad Sci 1187:129-139.
Cho E S, Johnson N, Snider B C (1984) Tissue glutathione as a cyst(e)ine reservoir during cystine depletion in growing rats. J Nutr 114:1853-1862.
Cotgreave I A (1997) N-acetylcysteine: pharmacological considerations and experimental and clinical applications. Adv Pharmacol 38:205-227.
Cregler L L, Mark H (1986) Cardiovascular dangers of cocaine abuse. Am J Cardiol 57:1185-1186.
Dackis C A, O'Brien C P (2001) Cocaine dependence: a disease of the brain's reward centers. J Subst Abuse Treat 21:111-117.
De Bernardi di Valserra M, Mautone G, Barindelli E, Lualdi P, Feletti F, Galmozzi M R (1989) Bioavailability of suckable tablets of oral N-acetylcysteine in man. Eur J Clin Pharmacol 37:419-421.
De Caro L, Ghizzi A, Costa R, Longo A, Ventresca G P, Lodola E (1989) Pharmacokinetics and bioavailability of oral acetylcysteine in healthy volunteers. Arzneimittelforschung 39:382-386.
Deves R, Boyd C A (2000) Surface antigen CD98(4F2): not a single membrane protein, but a family of proteins with multiple functions. J Membr Biol 173:165-177.
Dhakshinamoorthy S, Jaiswal A K (2002) c-Maf negatively regulates ARE-mediated detoxifying enzyme genes expression and anti-oxidant induction. Oncogene 21:5301-5312.
Dhakshinamoorthy S, Jain A K, Bloom D A, Jaiswal A K (2005) Bachl competes with Nrf2 leading to negative regulation of the antioxidant response element (ARE)-mediated NAD(P)H:quinone oxidoreductase 1 gene expression and induction in response to antioxidants. J Biol Chem 280:16891-16900.
Di Chiara G, Imperato A (1988) Drugs abused by humans preferentially increase synaptic dopamine concentrations in the mesolimbic system of freely moving rats. Proc Natl Acad Sci USA 85:5274-5278.
Di Chiara G, Bassareo V, Fenu S, De Luca M A, Spina L, Cadoni C, Acquas E, Carboni E, Valentini V, Lecca D (2004) Dopamine and drug addiction: the nucleus accumbens shell connection. Neuropharmacology 47 Suppl 1:227-241.
Dringen R, Hamprecht B (1999) N-acetylcysteine, but not methionine or 2-oxothiazolidine-4-carboxylate, serves as cysteine donor for the synthesis of glutathione in cultured neurons derived from embryonal rat brain. Neurosci Lett 259:79-82.
Everitt B J, Robbins T W (2005) Neural systems of reinforcement for drug addiction: from actions to habits to compulsion. Nat Neurosci 8:1481-1489.
Gabard B, Mascher H (1991) Endogenous plasma N-acetylcysteine and single dose oral bioavailability from two different formulations as determined by a new analytical method. Biopharm Drug Dispos 12:343-353.
Grandjean E M, Berthet P, Ruffmann R, Leuenberger P (2000a) Efficacy of oral long-term N-acetylcysteine in chronic bronchopulmonary disease: a meta-analysis of published double-blind, placebo-controlled clinical trials. Clin Ther 22:209-221.
Grandjean E M, Berthet P H, Ruffmann R, Leuenberger P (2000b) Cost-effectiveness analysis of oral N-acetylcysteine as a preventive treatment in chronic bronchitis. Pharmacol Res 42:39-50.
Grant J E, Kim S W, Odlaug B L (2007) N-Acetyl Cysteine, a Glutamate-Modulating Agent, in the Treatment of Pathological Gambling: A Pilot Study. Biological Psychiatry 62:652-657.
Gray K M, Watson N L, Carpenter M J, Larowe S D (2010) N-acetylcysteine (NAC) in young marijuana users: an open-label pilot study. Am J Addict 19:187-189.
Graybiel A M (2008) Habits, rituals, and the evaluative brain. Annu Rev Neurosci 31:359-387.
Greenslade R G, Mitchell S N (2004) Selective action of (+2-oxa-4-aminobicyclo[3.1.0]hexane-4,6-dicarboxylate (LY379268), a group II metabotropic glutamate receptor agonist, on basal and phencyclidine-induced dopamine release in the nucleus accumbens shell. Neuropharmacology 47:1-8.
Griffith O W (1999) Biologic and pharmacologic regulation of mammalian glutathione synthesis. Free Radic Biol Med 27:922-935.
Grueter B A, Winder D G (2005) Group II and III Metabotropic Glutamate Receptors Suppress Excitatory Synaptic Transmission in the Dorsolateral Bed Nucleus of the Stria Terminalis. Neuropsychopharmacology 30:1302-1311.
Heesch C M, Wilhelm C R, Ristich J, Adnane J, Bontempo F A, Wagner W R (2000) Cocaine activates platelets and increases the formation of circulating platelet containing microaggregates in humans. Heart 83:688-695.
Herrera-Marschitz M, You Z B, Goiny M, Meana J J, Silveira R, Godukhin O V, Chen Y, Espinoza S, Pettersson E, Loidl C F, Lubec G, Andersson K, Nylander I, Terenius L, Ungerstedt U (1996) On the origin of extracellular glutamate levels monitored in the basal ganglia of the rat by in vivo microdialysis. J Neurochem 66:1726-1735.
Holdiness M R (1991) Clinical pharmacokinetics of N-acetylcysteine. Clin Pharmacokinet 20:123-134.

Hu G, Duffy P, Swanson C, Ghasemzadeh M B, Kalivas P W (1999) The Regulation of Dopamine Transmission by Metabotropic Glutamate Receptors. J Pharmacol Exp Ther 289:412-416.

Jabaudon D, Shimamoto K, Yasuda-Kamatani Y, Scanziani M, Gahwiler B H, Gerber U (1999) Inhibition of uptake unmasks rapid extracellular turnover of glutamate of nonvesicular origin. Proc Natl Acad Sci USA 96:8733-8738.
Jaffe J H, Cascella N G, Kumor K M, Sherer M A (1989) Cocaine-induced cocaine craving. Psychopharmacology (Berl) 97:59-64.
Kalivas P W (2009) The glutamate homeostasis hypothesis of addiction. Nat Rev Neurosci 10:561-572.
Kalivas P W, Volkow N D (2005) The neural basis of addiction: a pathology of motivation and choice. Am J Psychiatry 162:1403-1413.
Kalivas P W, O'Brien C (2008) Drug addiction as a pathology of staged neuroplasticity. Neuropsychopharmacology 33:166-180.

Kalivas P W, Churchill L, Klitenick M A (1993) GABA and enkephalin projection from the nucleus accumbens and ventral pallidum to the ventral tegmental area. Neuroscience 57:1047-1060.
Karasawa J, Yoshimizu T, Chaki S (2006) A metabotropic glutamate 2/3 receptor antagonist, MGS0039, increases extracellular dopamine levels in the nucleus accumbens shell. Neurosci Lett 393:127-130.
Kau K S, Madayag A, Mantsch J R, Grier M D, Abdulhameed O, Baker D A (2008) Blunted cystine-glutamate antiporter function in the nucleus accumbens promotes cocaine-induced drug seeking. Neuroscience 155:530-537.
Knackstedt L A, Melendez R, Kalivas P W (2010) Ceftriaxone restores glutamate homeostasis and prevents relapse to cocaine seeking. Biol Psychiatry 67:81-84.
Knackstedt L A, LaRowe S, Mardikian P, Malcolm R, Upadhyaya H, Hedden S, Markou A, Kalivas P W (2009) The role of cystine-glutamate exchange in nicotine dependence in rats and humans. Biol Psychiatry 65:841-845.
Knuckey N R, Palm D, Primiano M, Epstein M H, Johanson C E (1995) N-acetylcysteine enhances hippocampal neuronal survival after transient forebrain ischemia in rats. Stroke 26:305-310; discussion 311.
Koob G F, Le Moal M (2001) Drug addiction, dysregulation of reward, and allostasis. Neuropsychopharmacology 24:97-129.
Kuczenski R, Segal D, Aizenstein M (1991) Amphetamine, cocaine, and fencamfamine: relationship between locomotor and stereotypy response profiles and caudate and accumbens dopamine dynamics. J Neuroscience 11(9):2703-2712.
La Bella V, Valentino F, Piccoli T, Piccoli F (2007) Expression and developmental regulation of the cystine/glutamate exchanger (xc-) in the rat. Neurochem Res 32:1081-1090.
LaRowe S D, Myrick H, Hedden S, Mardikian P, Saladin M, McRae A, Brady K, Kalivas P W, Malcolm R (2007) Is Cocaine Desire Reduced by N-Acetylcysteine? Am J Psychiatry 164:1115-1117.
Lee J M, Johnson J A (2004) An important role of Nrf2-ARE pathway in the cellular defense mechanism. J Biochem Mol Biol 37:139-143.
Lewerenz J, Maher P (2009) Basal levels of elF2alpha phosphorylation determine cellular antioxidant status by regulating ATF4 and xCT expression. J Biol Chem 284:1106-1115.
Lewerenz J, Klein M, Methner A (2006) Cooperative action of glutamate transporters and cystine/glutamate antiporter system Xc- protects from oxidative glutamate toxicity. J Neurochem 98:916-925.
Lo M, Ling V, Wang Y Z, Gout P W (2008) The xc- cystine/glutamate antiporter: a mediator of pancreatic cancer growth with a role in drug resistance. Br J Cancer 99:464-472.
Madayag A, Kau K S, Lobner D, Baker D A (2009) D1-like dopamine receptors on astrocytes regulate glutamate release: System xc- as a novel site for astrocyte-neuronal interactions
Madayag A, Lobner D, Kau K S, Mantsch J R, Abdulhameed O, Hearing M, Grier M D, Baker D A (2007) Repeated N-acetylcysteine administration alters plasticity-dependent effects of cocaine. J Neurosci 27:13968-13976.
Mann G E, Niehueser-Saran J, Watson A, Gao L, Ishii T, de Winter P, Siow R C (2007) Nrf2/ARE regulated antioxidant gene expression in endothelial and smooth muscle cells in oxidative stress: implications for atherosclerosis and preeclampsia. Sheng Li Xue Bao 59:117-127.
Manzoni O, Michel J M, Bockaert J (1997) Metabotropic glutamate receptors in the rat nucleus accumbens. Eur J Neurosci 9:1514-1523.
Manzoni O J, Williams J T (1999) Presynaptic regulation of glutamate release in the ventral tegmental area during morphine withdrawal. J Neurosci 19:6629-6636.
Marco C (2004) Distinct properties of presynaptic group II and III metabotropic glutamate receptor-mediated inhibition of perforant pathway&#x2013; CA1 EPSCs. European Journal of Neuroscience 19:2847-2858.
McBean G J (2002) Cerebral cystine uptake: a tale of two transporters. Trends Pharmacol Sci 23:299-302.
McLellan A T, Kushner H, Metzger D, Peters R, Smith I, Grissom G, Pettinati H, Argeriou M (1992) The Fifth Edition of the Addiction Severity Index. J Subst Abuse Treat 9:199-213.
Melendez R I, Vuthiganon J, Kalivas P W (2005) Regulation of extracellular glutamate in the prefrontal cortex: focus on the cystine glutamate exchanger and group I metabotropic glutamate receptors. J Pharmacol Exp Ther 314:139-147.
Mendelson J H, Mello N K (1996) Management of cocaine abuse and dependence. N Engl J Med 334:965-972.

Millea P J (2009) N-acetylcysteine: multiple clinical applications. Am Fam Physician 80:265-269.
Miller L F, Rumack B H (1983) Clinical safety of high oral doses of acetylcysteine. Semin Oncol 10:76-85.
Montague P R (2008) Free will. Curr Biol 18:R584-585.
Montana M C, Cavallone L F, Stubbert K K, Stefanescu A D, Kharasch E D, Gereau R Wt (2009) The metabotropic glutamate receptor subtype 5 antagonist fenobam is analgesic and has improved in vivo selectivity compared with the prototypical antagonist 2-methyl-6-(phenylethynyl)-pyridine. J Pharmacol Exp Ther 330:834-843.
Moran M M, McFarland K, Melendez R I, Kalivas P W, Seamans J K (2005) Cystine/glutamate exchange regulates metabotropic glutamate receptor presynaptic inhibition of excitatory transmission and vulnerability to cocaine seeking. J Neurosci 25:6389-6393.
Ohishi H, Shigemoto R, Nakanishi S, Mizuno N (1993a) Distribution of the mRNA for a metabotropic glutamate receptor (mGluR3) in the rat brain: an in situ hybridization study. J Comp Neurol 335:252-266.
Ohishi H, Shigemoto R, Nakanishi S, Mizuno N (1993b) Distribution of the messenger RNA for a metabotropic glutamate receptor, mGluR2, in the central nervous system of the rat. Neuroscience 53:1009-1018.
Olsson B, Johansson M, Gabrielsson J, Bolme P (1988) Pharmacokinetics and bioavailability of reduced and oxidized N-acetylcysteine. Eur J Clin Pharmacol 34:77-82.
Patel S A, Warren B A, Rhoderick J F, Bridges R J (2004) Differentiation of substrate and non-substrate inhibitors of transport system xc(−): an obligate exchanger of L-glutamate and L-cystine. Neuropharmacology 46:273-284.
Poisik O, Raju D V, Verreault M, Rodriguez A, Abeniyi O A, Conn P J, Smith Y (2005) Metabotropic glutamate receptor 2 modulates excitatory synaptic transmission in the rat globus pallidus. Neuropharmacology 49:57-69.
Pow D V (2001) Visualising the activity of the cystine-glutamate antiporter in glial cells using antibodies to aminoadipic acid, a selectively transported substrate. Glia 34:27-38.
Prescott L F, Park J, Ballantyne A, Adriaenssens P, Proudfoot A T (1977) Treatment of paracetamol (acetaminophen) poisoning with N-acetylcysteine. Lancet 2:432-434.
Richards G, Messer J, Malherbe P, Pink R, Brockhaus M, Stadler H, Wichmann J, Schaffhauser H, Mutel V (2005) Distribution and abundance of metabotropic glutamate receptor subtype 2 in rat brain revealed by [3H]LY354740 binding in vitro and quantitative radioautography: correlation with the sites of synthesis, expression, and agonist stimulation of [35 S]GTPgammas binding. J Comp Neurol 487:15-27.
Roes E M, Raijmakers M T, Peters W H, Steegers E A (2002) Effects of oral N-acetylcysteine on plasma homocysteine and whole blood glutathione levels in healthy, non-pregnant women. Clin Chem Lab Med 40:496-498.
Sadowska A M, Manuel Y K B, De Backer W A (2007) Antioxidant and anti-inflammatory efficacy of NAC in the treatment of COPD: discordant in vitro and in vivo dose-effects: a review. Pulm Pharmacol Ther 20:9-22.
Sagara J I, Miura K, Bannai S (1993) Maintenance of neuronal glutathione by glial cells. J Neurochem 61:1672-1676.
Sasaki H, Sato H, Kuriyama-Matsumura K, Sato K, Maebara K, Wang H, Tamba M, Itoh K, Yamamoto M, Bannai S (2002) Electrophile response element-mediated induction of the cystine/glutamate exchange transporter gene expression. J Biol Chem 277:44765-44771.
Sato H, Fujiwara K, Sagara J, Bannai S (1995) Induction of cystine transport activity in mouse peritoneal macrophages by bacterial lipopolysaccharide. Biochem J 310 (Pt 2):547-551.
Sato H, Tamba M, Ishii T, Bannai S (1999) Cloning and expression of a plasma membrane cystine/glutamate exchange transporter composed of two distinct proteins. J Biol Chem 274:11455-11458.
Sato H, Tamba M, Okuno S, Sato K, Keino-Masu K, Masu M, Bannai S (2002) Distribution of cystine/glutamate exchange transporter, system x(c)-, in the mouse brain. J Neurosci 22:8028-8033.
Schoenbaum G, Roesch M R, Stalnaker T A (2006) Orbitofrontal cortex, decision-making and drug addiction. Trends Neurosci 29:116-124.
Schoepp D D (2001) Unveiling the Functions of Presynaptic Metabotropic Glutamate Receptors in the Central Nervous System. J Pharmacol Exp Ther 299:12-20.
Scholze A, Rinder C, Beige J, Riezler R, Zidek W, Tepel M (2004) Acetylcysteine reduces plasma homocysteine concentration and improves pulse pressure and endothelial function in patients with end-stage renal failure. Circulation 109:369-374.
Shih A Y, Erb H, Sun X, Toda S, Kalivas P W, Murphy T H (2006) Cystine/glutamate exchange modulates glutathione supply for neuroprotection from oxidative stress and cell proliferation. J Neurosci 26:10514-10523.
Siegel A J, Sholar M B, Mendelson J H, Lukas S E, Kaufman M J, Renshaw P F, McDonald J C, Lewandrowski K B, Apple F S, Stec J J, Lipinska I, Tofler G H, Ridker P M (1999) Cocaine-induced erythrocytosis and increase in von Willebrand factor: evidence for drug-related blood doping and prothrombotic effects. Arch Intern Med 159:1925-1929.
Singer L T, Arendt R, Minnes S, Farkas K, Salvator A, Kirchner H L, Kliegman R (2002) Cognitive and motor outcomes of cocaine-exposed infants. JAMA 287:1952-1960.
Sjodin K, Nilsson E, Hallberg A, Tunek A (1989) Metabolism of N-acetyl-L-cysteine. Some structural requirements for the deacetylation and consequences for the oral bioavailability. Biochem Pharmacol 38:3981-3985.
Smilkstein M J, Knapp G L, Kulig K W, Rumack B H (1988) Efficacy of oral N-acetylcysteine in the treatment of acetaminophen overdose. Analysis of the national multicenter study (1976 to 1985). N Engl J Med 319:1557-1562.
Tamaru Y, Nomura S, Mizuno N, Shigemoto R (2001) Distribution of metabotropic glutamate receptor mGluR3 in the mouse CNS: differential location relative to pre- and postsynaptic sites. Neuroscience 106:481-503.
Tang X C, Kalivas P W (2003) Bidirectional modulation of cystine/glutamate exchanger activity in cultured cortical astrocytes. Ann N Y Acad Sci 1003:472-475.
Testa C M, Friberg I K, Weiss S W, Standaert D G (1998) Immunohistochemical localization of metabotropic glutamate receptors mGluR1a and mGluR2/3 in the rat basal ganglia. J Comp Neurol 390:5-19.

Timmerman W, Westerink B H (1997) Brain microdialysis of GABA and glutamate: what does it signify? Synapse 27:242-261.
Ventura P, Panini R, Abbati G, Marchetti G, Salvioli G (2003) Urinary and plasma homocysteine and cysteine levels during prolonged oral N-acetylcysteine therapy. Pharmacology 68:105-114.
Warr O, Takahashi M, Attwell D (1999) Modulation of extracellular glutamate concentration in rat brain slices by cystine-glutamate exchange. J Physiol 514 (Pt 3):783-793.
Westerink B H (1995) Brain microdialysis and its application for the study of animal behaviour. Behav Brain Res 70:103-124.
Xi Z-X, Baker D A, Shen H, Carson D S, Kalivas P W (2002) Group II Metabotropic Glutamate Receptors Modulate Extracellular Glutamate in the Nucleus Accumbens. J Pharmacol Exp Ther 300:162-171.
Xi Z X, Kiyatkin M, Li X, Peng X Q, Wiggins A, Spiller K, Li J, Gardner E L (2010) N-acetylaspartylglutamate (NAAG) inhibits intravenous cocaine self-administration and cocaine-enhanced brain-stimulation reward in rats. Neuropharmacology 58:304-313.
Xie X, Steketee J D (2008) Repeated exposure to cocaine alters the modulation of mesocorticolimbic glutamate transmission by medial prefrontal cortex Group II metabotropic glutamate receptors. J Neurochem 107:186-196.
Yamauchi A, Ueda N, Hanafusa S, Yamashita E, Kihara M, Naito S (2002) Tissue distribution of and species differences in deacetylation of N-acetyl-L-cysteine and immunohistochemical localization of acylase I in the primate kidney. J Pharm Pharmacol 54:205-212.
Zhou W, Kalivas P W (2008) N-acetylcysteine reduces extinction responding and induces enduring reductions in cue- and heroin-induced drug-seeking. Biol Psychiatry 63:338-340.

We claim:

1. A method of treating subjects comprising, administering a metabotropic glutamate receptor (mGluR) modulator and a procysteine drug, wherein the subject has had a prior addiction

2. The method of claims 1-35, wherein the prior addiction is a drug addiction.

3. The method of claim 2, wherein the drug addiction is cocaine addiction

4. The method of claims 1-35, wherein the mGluR modulator is a negative modulator.

5. The method of claims 4, wherein the negative modulator is a negative allosteric modulator.

6. The method of claims 1-35, wherein the combination of these compounds can reduce drug use.

7. The method of claim 1-35 wherein the mGluR is mGluR5.

8. The method of claims 1-35, wherein the mGluR5 modulator is 2-methyl-6-(phenylethynyl)pyridine (MPEP).

9. The method of claims 1-35, wherein the mGluR5 modulator is 3-((2-Methyl-4-thiazolyl)ethynyl)pyridine (MTEP).

10. The method of claims 1-35 wherein the procysteine drug is N-acetylcysteine (NAC)

11. The method of claims 1-35 wherein the mGluR modulator is administered at subthreshold levels.

12. The method of claims 1-35 wherein the procysteine drug is administered at subthreshold levels.

13. The method of claims 1-35 wherein the mGluR modulator and procysteine drug are both administered at subthreshold levels.

14. The method of claims 1-35, wherein the mGluR modulator and procysteine drug are each administered at therapeutic levels.

15. The method of claims 1-35, wherein the mGluR modulator and procysteine drug have a synergistic effect.

16. The method of claims 1-35, wherein the NAC activates mGluR2/3

17. The method of claims 1-35, wherein the mGluR modulator and procysteine drug are administered simultaneously.

18. The method of claims 1-35, wherein the mGluR modulator and procysteine drug are administered consecutively.

19. The method of claims 1-35 wherein the mGluR modulator and procysteine drug are administered before the subject encounters a drug cue.

20. The method of claims 1-35, wherein the mGluR modulator and procysteine drug are administered intraperitoneally.

21. The method of claims 1-35, wherein the mGluR modulator and procysteine drug are administered in one composition.

22. The method of claims 1-35, wherein side effects are decreased in subjects administered both the mGluR modulator and procysteine drug compared to subjects administered either of them alone

23. A method of inhibiting drug seeking comprising:
a) identifying a subject at risk for drug use; and
b) administering a mGluR modulator and a procysteine drug to the subject.

24. A method of preventing drug use in a subject comprising:
a) identifying the subject as being at risk for drug use;
b) administering a mGluR modulator and a procysteine drug to the subject

25. A method of decreasing glutamate release in the neuronal synapse and glutamate binding to mGluR5 comprising administering a mGluR modulator and a procysteine drug to a subject at risk for drug use.

26. The method of claim 25, wherein the drug use is cocaine use.

27. The method of claims 1-26, wherein the mGluR modulator has the structure of: 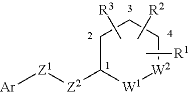
wherein the six membered ring defined by W¹, W² and carbon atoms 1, 2, 3 and 4, can be aromatic or non-aromatic, and further wherein any two neighboring atoms of this six membered ring may be singly or doubly bonded to one another;
Z¹ and Z² are either carbon or nitrogen, further wherein Z¹ and Z² can be singly, doubly, or triply bonded to one another and wherein Z² and carbon atom 1 can be singly or doubly bonded to one another, provided that the bond between Z¹ and Z² is not triple when Z₁ and Z₂ are nitrogen, further provided that the bond between Z¹ and Z² is single when the bond between Z² and carbon atom 1 is double;
Ar is substituted or unsubstituted aryl or substituted or unsubstituted heteroaryl;
one of either W¹ and W² is nitrogen and the other is carbon;
R¹, R², and R³ are independently hydrogen, hydroxy, amino, cyano, halo, nitro, mercapto, or a heteroatom-substituted or heteroatom-unsubstituted alkyl, alkenyl, alkynyl, aryl, aralkyl, acyl, alkoxy, alkylamino, or =0; or pharmaceutically acceptable salts, hydrates, tautomers, acetals, ketals, hemiacetals, hemiketals, or optical isomers thereof.

28. The method of claim 27, wherein Z₁ and Z₂ are both carbon triply bonded to each other.

29. The method of claim 27, wherein Z₁ and Z₂ are both nitrogen doubly bonded to each other.

30. The method of claim 27, wherein Ar is phenyl, substituted phenyl, thiazole or substituted thiazole.

31. The method of claim 27, wherein W¹ is nitrogen and W² is carbon.

32. The method of claim 27, wherein W² is nitrogen and W¹ is carbon.

33. The method of claim 27, wherein R¹, R², and R³ are independently hydrogen, amino, alkyl, alkenyl or halo.

34. The method of claim 27, wherein the mGluR modulator is: phenazopyridine; SIB 1893; SIB 1757; 2-methyl-6-(phenylethynyl)-pyridine (MPEP); NSC41777; 6-methyl-3-phenyldiazenylpyridin-2-amine; 2,6-Diamino-3-(4-iodophenylazo)pyridine; phenyldiazenylpyridin-2-amine; 3-(4-chlorophenyl)diazenylpyridine-2,6-diamine; 3-(2-chlorophenyl)diazenylpyridine-2,6-diamine; 3-[(2-methyl-1,3-thiazol-4-yl)ethynyl]pyridine (MTEP); 1-(3-chlorophenyl)-3-(3-methyl-5-oxo-4H-imidazol-2-yl)urea; 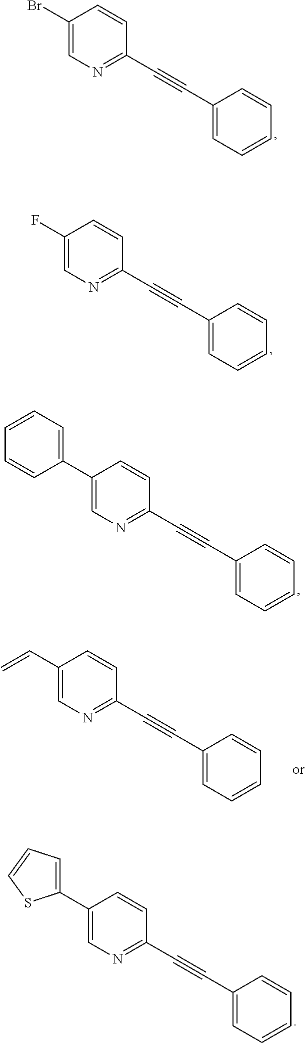
a physiologically acceptable salt thereof; or any mixture thereof.

35. The method of claim 27-34, wherein the procysteine drug is NAC, DiNAC; N-acetylcysteine L-lysine; Carbocisteine; glutathione; S-nitroso-N-acetylcysteine; S-nitrosothiol-N-acetylcysteine; S-allyl-cysteine; S-alkyl-cysteine; N-acetyl-S-farnesyl-cysteine; N-acetyl-L-arginine-NAC; N-acetyl-L-lysine-NAC; N-acetyl-L-histidine-NAC; N-acetyl-L-ornithine-NAC; thioester of NAC with salicylic acid; 2′4′-difluoro-4-hydroxy-(1,1′-diphenyl)-3-carboxylic derivatives of NAC; S-allymercapto-NAC (ASSNaC); N,N-diacetyl-L-cystine; N—S-diacyl-cysteine; N-acetylcysteine conjugate of phenethyl isothiocyanate (PEITC-NAC); S-carboxylmethyl-L-cysteine; derivatives of reacting a reactive derivative of p-isobutylphenylpropionic acid and NAC (e.g., an amide); paraisobutyl NAC; L-2-oxothiazolidine-4-carboxylic acid and a combination thereof.

36. The method of claim 27-34, wherein the procysteine drug have the structure: 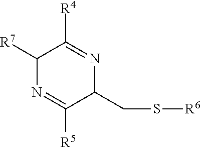
wherein: R⁴ and R⁵ are independently selected from OH, ═O, or a branched or straight chain C₁ to C₅ alkoxyl group, with the caveat that when =0 is selected the nitrogen atom adjacent the carbonyl group thusly formed bears a H and a single bond joins the adjacent nitrogen to said carbonyl group;
R⁶ is H, a branched or straight chain C₁ to C₅ alkyl, a nitrobenzenesulfonyl, a trityl, an aryl thio, an aryl, an alkylthio, an acyl, a benzoyl, a thio acyl, a thio benzoyl, or a benzyl group;
R⁷ is selected from the side chain groups of the natural L-amino acids cys, gly, phe, pro, val, ser, arg, asp, asn, glu, gin, ala, his, ile, leu, lys, met, thr, trp, tyr, or D-isomers thereof, with the caveat that when R⁴ is the side chain group of the natural L-amino acid gly, R¹ and R² are not both selected to be ═O; or a cystine dimer of said drug having the structure: 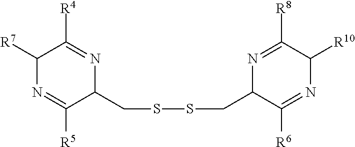
wherein: R⁴, R⁵, R⁸ and R⁹ are independently selected from OH, ═O, or a branched or straight chain C₁ to C₅ alkoxyl group, with the caveat that when =0 is selected the nitrogen atom adjacent the carbonyl group thusly formed bears a H and a single bond joins the adjacent nitrogen to said carbonyl group; and
R⁷ and R¹⁰ are independently selected from the side chain groups of the natural L-amino acids cys, gly, phe, pro, val, ser, arg, asp, asn, glu, gin, ala, his, ile, leu, lys, met, thr, trp, tyr, or D-isomers thereof, with the caveat that when R⁷ and R¹⁰ are both the side chain group of the natural L-amino acid gly, R⁴, R⁵, R⁸ and R⁹ shall not all be selected to be ═O.

37. The method of claim 36, wherein the cysteine drug have the structure:

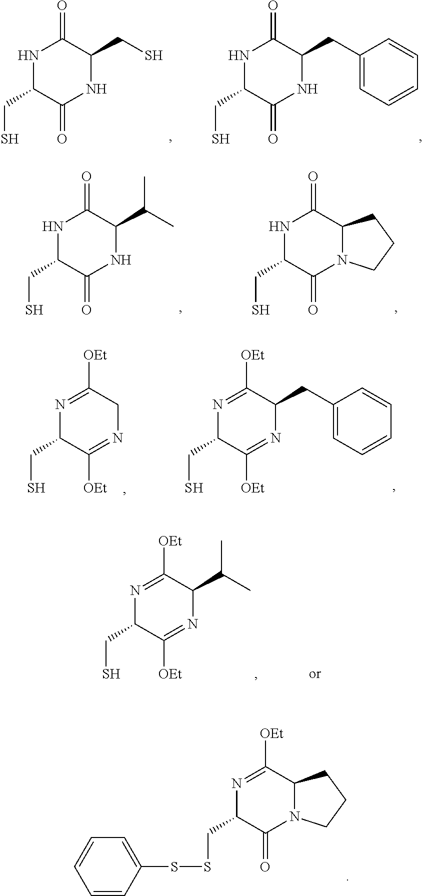

38. The method of claim 36, wherein the cysteine drug is in the form of a cysteine dimer having the structure:

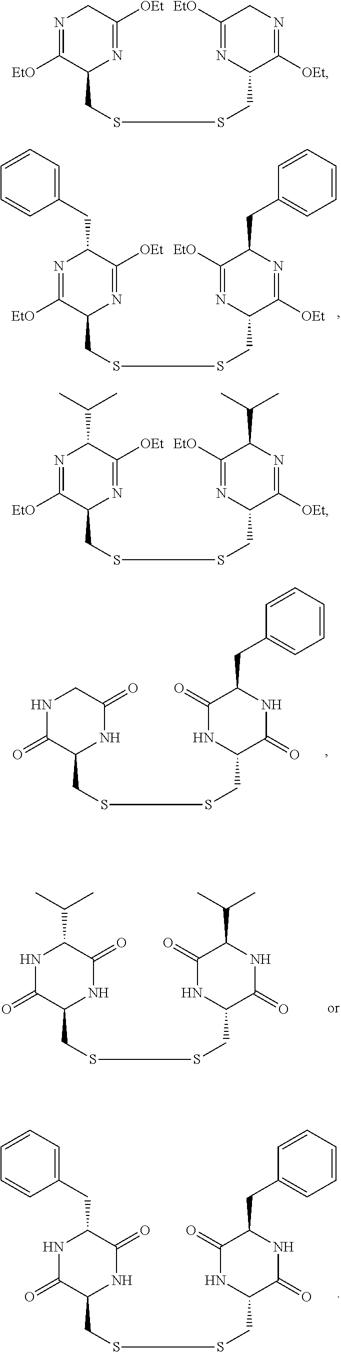

39. The method of claim 36, wherein the cysteine drug is in the form of a cysteine dimer and R⁷ and R¹⁰ are identical.

40. The method of claim 36, wherein the cysteine drug is in the form of a cystine dimer and R⁷ and R¹⁰ are non-identical.

41. The method of claim 36, wherein the cysteine drug or cystine dimer thereof includes at least one R⁷ and R¹⁰ group that is a cys, said cys is further protected by a branched or straight chain C₁ to C₅ alkyl, a nitrobenzenesulfonyl, a trityl, an aryl thio, an aryl, an alkylthio, an acyl, a benzoyl, a thio acyl, a thio benzoyl, or a benzyl group.

42. The method of claim 36, wherein the cysteine drug have the structure: 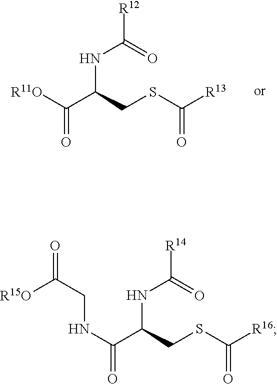
or a cystine dimer of the cystein drug that have the structure: 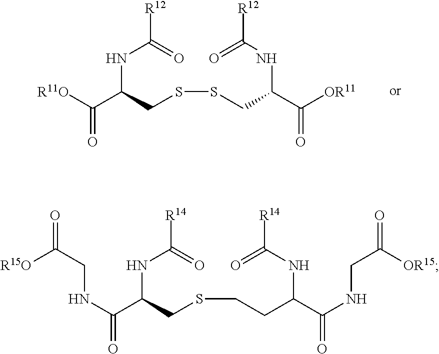
wherein R¹¹ through R¹⁶ are independently selected from a branched or straight chain C₁ to C₅ alkyl, a phenyl, or a benzyl group.

43. The method of claim 42, wherein the cysteine drug have the structure:

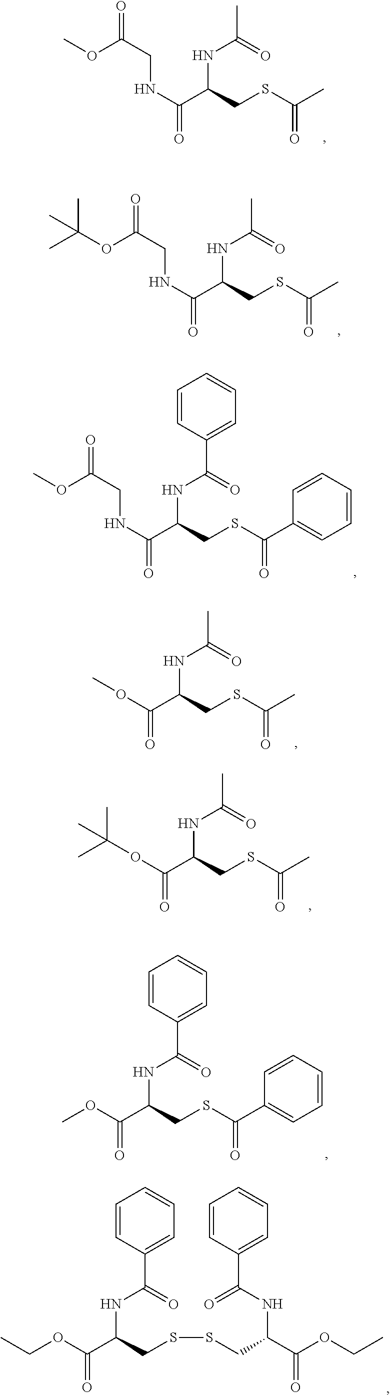

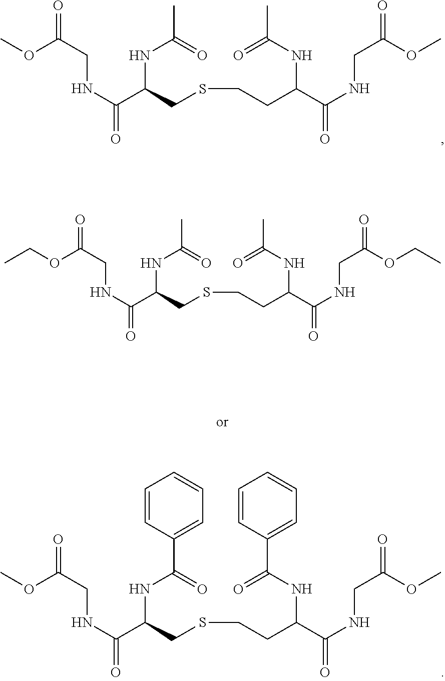

44. A composition comprising a mGluR modulator and procysteine drug.

45. The composition of claims 44-73, wherein the mGluR modulator has the structure: 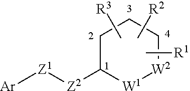
wherein the six membered ring defined by W¹, W² and carbon atoms 1, 2, 3 and 4, can be aromatic or non-aromatic, and further wherein any two neighboring atoms of this six membered ring may be singly or doubly bonded to one another;
Z¹ and Z² are either carbon or nitrogen, further wherein Z¹ and Z² can be singly, doubly, or triply bonded to one another and wherein Z² and carbon atom 1 can be singly or doubly bonded to one another, provided that the bond between Z¹ and Z² is not triple when Z₁ and Z₂ are nitrogen, further provided that the bond between Z¹ and Z² is single when the bond between Z² and carbon atom 1 is double;
Ar is substituted or unsubstituted aryl or substituted or unsubstituted heteroaryl;
one of either W¹ and W² is nitrogen and the other is carbon;
R¹, R², and R³ are independently hydrogen, hydroxy, amino, cyano, halo, nitro, mercapto, or a heteroatom-substituted or heteroatom-unsubstituted alkyl, alkenyl, alkynyl, aryl, aralkyl, acyl, alkoxy, alkylamino, or =0; or pharmaceutically acceptable salts, hydrates, tautomers, acetals, ketals, hemiacetals, hemiketals, or optical isomers thereof.

46. The composition of claims 44-73, wherein Z₁ and Z₂ are both carbon triply bonded to each other.

47. The composition of claims 44-73, wherein Z₁ and Z₂ are both nitrogen doubly bonded to each other.

48. The composition of claims 44-73, wherein Ar is phenyl, substituted phenyl, thiazole or substituted thiazole.

49. The composition of claims 44-73, wherein W¹ is nitrogen and W² is carbon.

50. The composition of claims 44-73, wherein W² is nitrogen and W¹ is carbon.

51. The composition of claims 44-73, wherein R¹, R², and R³ are independently hydrogen, amino, alkyl, alkenyl or halo.

52. The composition of claims 44-73, wherein the mGluR modulator is: phenazopyridine; SIB 1893; SIB 1757; 2-methyl-6-(phenylethynyl)-pyridine (MPEP); NSC41777; 6-methyl-3-phenyldiazenylpyridin-2-amine; 2,6-Diamino-3-(4-iodophenylazo)pyridine; phenyldiazenylpyridin-2-amine; 3-(4-chlorophenyl)diazenylpyridine-2,6-diamine; 3-(2-chlorophenyl)diazenylpyridine-2,6-diamine; 3-[(2-methyl-1,3-thiazol-4-yl)ethynyl]pyridine (MTEP); 1-(3-chlorophenyl)-3-(3-methyl-5-oxo-4H-imidazol-2-yl)urea; 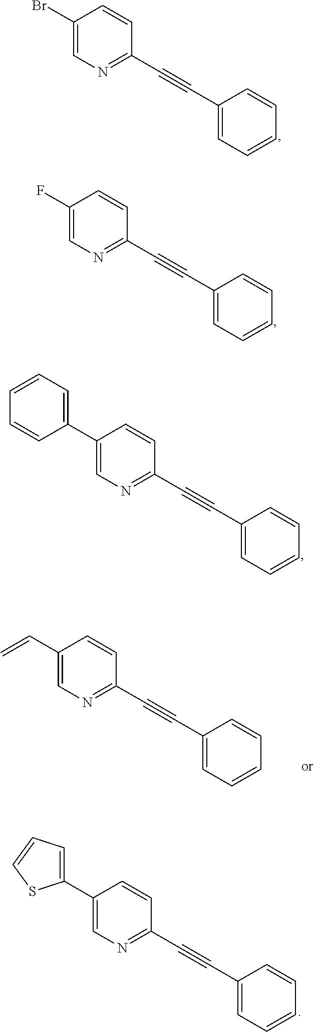
a physiologically acceptable salt thereof; or any mixture thereof.

53. The method of claim 44-72, wherein the procysteine drug is NAC, DiNAC; N-acetylcysteine L-lysine; Carbocisteine; glutathione; S-nitroso-N-acetylcysteine; S-nitrosothiol-N-acetylcysteine; S-allyl-cysteine; S-alkyl-cysteine; N-acetyl-S-farnesyl-cysteine; N-acetyl-L-arginine-NAC; N-acetyl-L-lysine-NAC; N-acetyl-L-histidine-NAC; N-acetyl-L-ornithine-NAC; thioester of NAC with salicylic acid; 2′4′-difluoro-4-hydroxy-(1,1′-diphenyl)-3-carboxylic derivatives of NAC; S-allymercapto-NAC (ASSNaC); N,N-diacetyl-L-cystine; N—S-diacyl-cysteine; N-acetylcysteine conjugate of phenethyl isothiocyanate (PEITC-NAC); S-carboxylmethyl-L-cysteine; derivatives of reacting a reactive derivative of p-isobutylphenylpropionic acid and NAC (e.g., an amide); paraisobutyl NAC; L-2-oxothiazolidine-4-carboxylic acid and a combination thereof.

54. The composition of claim 44-73, wherein the procysteine drug have the structure: 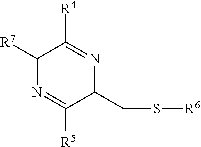
wherein: R⁴ and R⁵ are independently selected from OH, ═O, or a branched or straight chain C₁ to C₅ alkoxyl group, with the caveat that when =0 is selected the nitrogen atom adjacent the carbonyl group thusly formed bears a H and a single bond joins the adjacent nitrogen to said carbonyl group;
R⁶ is H, a branched or straight chain C₁ to C₅ alkyl, a nitrobenzenesulfonyl, a trityl, an aryl thio, an aryl, an alkylthio, an acyl, a benzoyl, a thio acyl, a thio benzoyl, or a benzyl group;
R⁷ is selected from the side chain groups of the natural L-amino acids cys, gly, phe, pro, val, ser, arg, asp, asn, glu, gin, ala, his, ile, leu, lys, met, thr, trp, tyr, or D-isomers thereof, with the caveat that when R⁴ is the side chain group of the natural L-amino acid gly, R¹ and R² are not both selected to be ═O; or a cystine dimer of said drug having the structure: 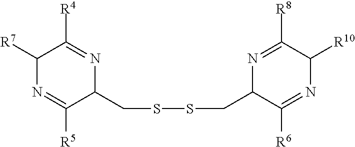
wherein: R⁴, R⁵, R⁸ and R⁹ are independently selected from OH, ═O, or a branched or straight chain C₁ to C₅ alkoxyl group, with the caveat that when =0 is selected the nitrogen atom adjacent the carbonyl group thusly formed bears a H and a single bond joins the adjacent nitrogen to said carbonyl group; and
R⁷ and R¹⁶ are independently selected from the side chain groups of the natural L-amino acids cys, gly, phe, pro, val, ser, arg, asp, asn, glu, gin, ala, his, ile, leu, lys, met, thr, trp, tyr, or D-isomers thereof, with the caveat that when R⁷ and R¹⁰ are both the side chain group of the natural L-amino acid gly, R⁴, R⁵, R⁸ and R⁹ shall not all be selected to be ═O.

54. The composition of claim 44-73, wherein the cysteine drug have the structure:

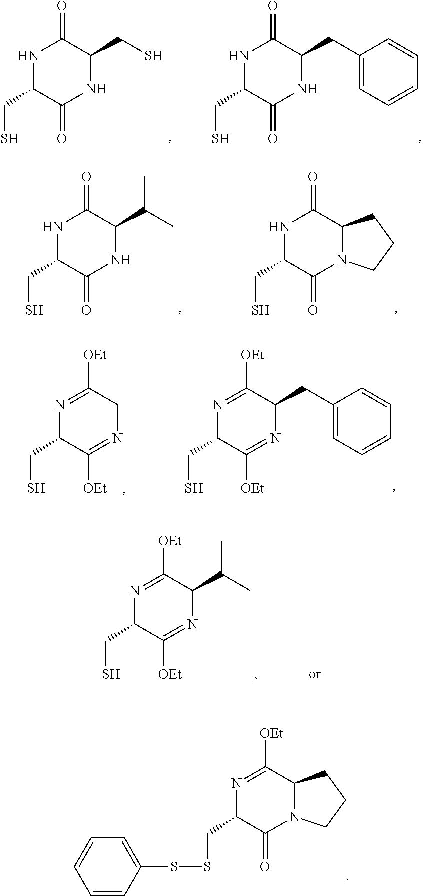

56. The composition of claim 44-73, wherein the cysteine drug is in the form of a cysteine dimer having the structure:

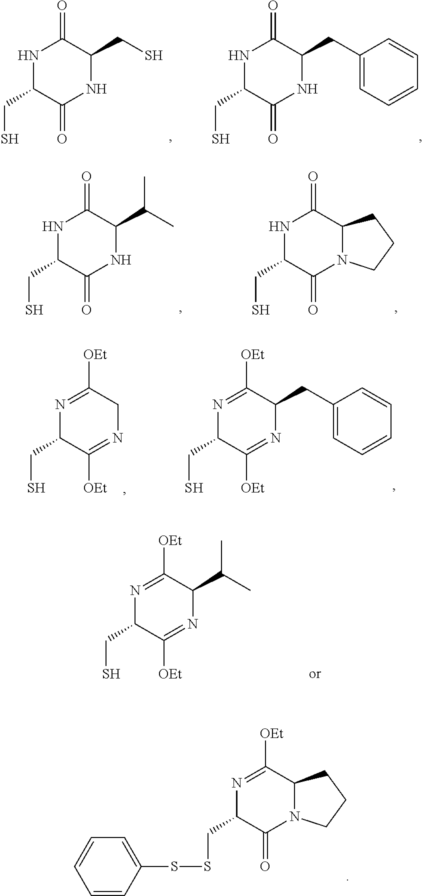

57. The composition of claim 44-73, wherein the cysteine drug is in the form of a cysteine dimer and R⁷ and R¹⁰ are identical.

58. The composition of claim 44-73, wherein the cysteine drug is in the form of a cystine dimer and R⁷ and R¹⁰ are non-identical.

59. The composition of claim 44-73, wherein the cysteine drug or cystine dimer thereof includes at least one R⁷ and R¹⁰ group that is a cys, said cys is further protected by a branched or straight chain C₁ to C₅ alkyl, a nitrobenzenesulfonyl, a trityl, an aryl thio, an aryl, an alkylthio, an acyl, a benzoyl, a thio acyl, a thio benzoyl, or a benzyl group.

60. The composition of claim 44-73, wherein the cysteine drug have the structure: 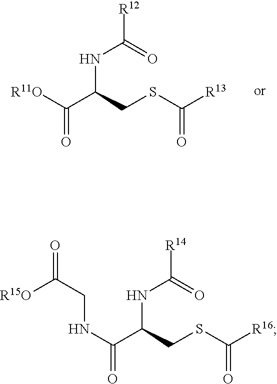
or a cystine dimer of the cystein drug that have the structure: 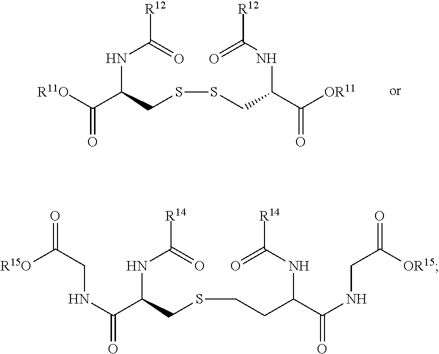
wherein R¹¹ through R¹⁶ are independently selected from a branched or straight chain C1 to C5 alkyl, a phenyl, or a benzyl group.

61. The composition of claim 44-73, wherein the cysteine drug have the structure:

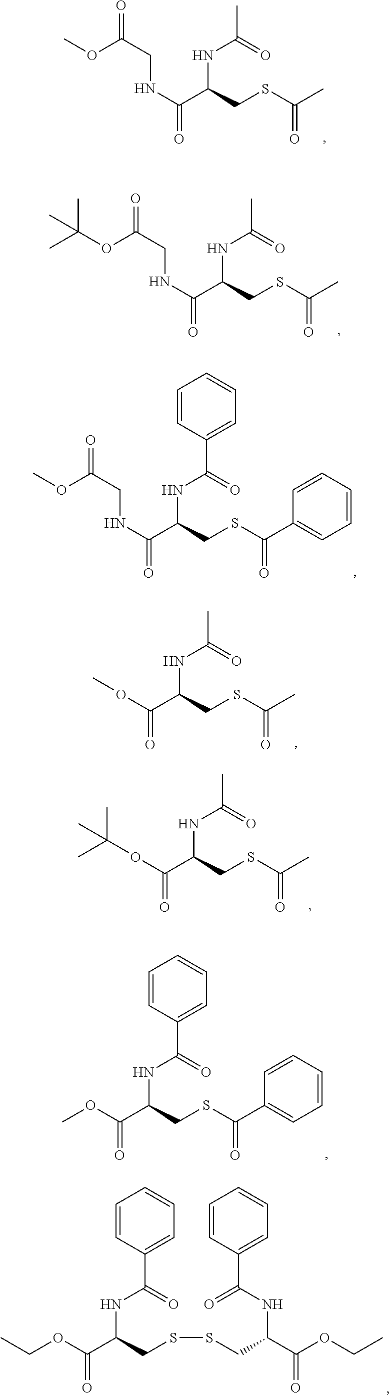

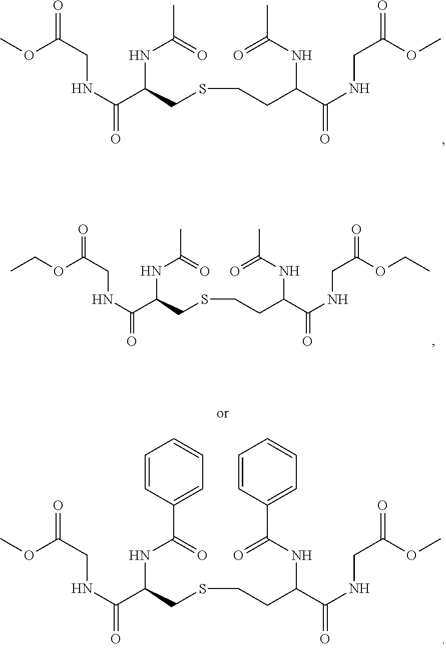

62. The composition of claims 44-73, wherein the composition reduces drug use.

63. The composition of claims 44-73, wherein the mGluR modulator and procysteine drug are each at subthreshold levels.

64. The composition of claims 44-73, wherein the mGluR modulator and procysteine drug are each at therapeutic levels.

65. The composition of claims 44-73, wherein the mGluR modulator is at subthreshold levels and the procysteine drug is at therapeutic levels.

66. The composition of claims 44-73, wherein the mGluR modulator is at therapeutic levels and the procysteine drug is at subthreshold levels.

67. The composition of claims 44-73, wherein side effects are decreased compared to therapeutic levels of either compound alone.

68. The composition of claims 44-73, wherein the mGluR modulator is a mGluR5 modulator.

69. The composition of claims 44-73, wherein the mGluR5 modulator is a negative modulator.

70. The composition of claim 69, wherein the negative modulator is a negative allosteric modulator.

71. The composition of claims 44-73, wherein the procysteine drug is NAC.

72. The composition of claims 44-73, wherein the mGluR5 modulator is 342-Methyl-4-thiazolyl)ethynyl)pyridine (MPEP).

73. The composition of claims 44-73, wherein the drug addiction is cocaine addiction.
